(19) United States
(12) Patent Application Publication
(10) Pub. No.: US 2026/0162247 A1
(43) Pub. Date: Jun. 11, 2026

(54) QA SYSTEM AND METHOD

(71) Applicant: JBT Marel Corporation, Chicago, IL (US)

(72) Inventors: Jon A. Hocker, Redmond, WA (US); Richard D. Stockard, San Diego, CA (US); Alec W.W. Hewitt, Loveland, CO (US); George R. Blaine, Lake Stevens, WA (US); Jeffrey William Worley, Seattle, WA (US)

(73) Assignee: JBT Marel Corporation, Chicago, IL (US)

(21) Appl. No.: 19/150,132

(22) PCT Filed: Jan. 23, 2024
(86) PCT No.: PCT/US2024/012617

§ 371 Date: Jul. 22, 2025

Related U.S. Application Data
(60) Provisional application No. 63/481,113, filed on Jan. 23, 2023.

(51) Int. Cl. G06T 7/00; G01N 9/36; G01N 33/02; G06T 7/90; G06T 17/00

(52) U.S. Cl. CPC G06T 7/001; G01N 9/36; G01N 33/02; G06T 7/90; G06T 17/00; G06T 2200/04; G06T 2207/10024; G06T 2207/30128; G06T 2207/30164

(57) ABSTRACT

A computer-implemented method of performing a quality assurance (QA) analysis for a QA sample may include capturing image sensor data of a QA sample with an image sensor assembly; generating, with a computing device, at least one of a 2D model and a 3D model of the QA sample using the image sensor data; and performing, with a computing device, a QA analysis of the QA sample by comparing data points of the at least one of the 2D model and 3D model of the QA sample and a corresponding 2D model and 3D model from a QA specification of the QA sample.

### CROSS-REFERENCE TO RELATED APPLICATION

This application claims the benefit of U.S. Provisional Application No. 63/481,113, filed Jan. 23, 2023, the entire contents of which are incorporated herein by reference.

### BACKGROUND

Workpieces, including food products, are portioned or otherwise cut into smaller pieces by processors in accordance with customer needs. Also, excess fat, bones, and other foreign or undesired materials are routinely trimmed from food products. It is usually highly desirable to portion and/or trim the food products into uniform sizes, for example, for steaks to be served at restaurants or chicken fillets used in frozen dinners or in chicken burgers.

Much of the portioning/trimming of workpieces, in particular food products, is now carried out with the use of high-speed portioning machines. These machines use various scanning techniques to ascertain the size and shape of the food product as it is being advanced on a moving conveyor. This information is analyzed with the aid of a computer to determine how to most efficiently portion the food product into optimum sizes. For example, a customer may desire chicken breast portions in two different weight sizes, but with no fat or with a limited amount of acceptable fat. The chicken breast is scanned as it moves on an infeed conveyor belt and a determination is made through the use of a computer as to how best to portion the chicken breast to the weights desired by the customer, with no or limited amount of fat, so as to use the chicken breast most effectively.

Portioning and/or trimming of workpieces can be carried out by various cutting devices, including high-speed liquid jet cutters (liquids may include, for example, water or liquid nitrogen) or rotary or reciprocating blades, after the food product is transferred from the infeed to a cutting conveyor. In many high-speed portioning systems, several high-speed waterjet cutters are positioned along the length of a conveyor to achieve high throughput of the portioned/cut workpieces. Once the portioning/trimming has occurred, the resulting portions are off-loaded from the cutting conveyor and placed on a take-away conveyor for further processing or, perhaps, to be placed in a storage bin.

Although the high-speed portioning machines referenced herein are highly sophisticated for analyzing workpieces and for determining how to optimally portion or cut such workpieces at high production rates (e.g., typically over 200 pieces per minute), variations in shapes, dimensions, weights, densities, colors, and textures of incoming, raw, unprocessed food products cannot always be accounted for. Moreover, even if the portioner machines are frequently re-calibrated, the machine can quickly become out of sync (e.g., due to component wear, timing issues, etc.)

Accordingly, the difference between correct, intended portioning cuts and incorrect portioning cuts may be subtle and frequent on one hand, or may be dramatic and rare on the other hand. In either situation, the cutting error may be “hidden” among the many thousands of pieces of food products being portioned per hour. Even trained, observant operators, watching for specific problems, for example, too heavy or too lightweight portions, may have difficulty spotting “outliers,” especially since the portions pass by on a conveyor belt at a speed of two to three pieces per second for larger pieces, or at a speed of about 10 pieces per second for smaller pieces. Moreover, inaccuracies in portioning often develop slowly over time, and thus may be difficult for operators to notice.

### SUMMARY

In some aspects, the techniques described herein relate to a computer-implemented method of performing a quality assurance (QA) analysis for a QA sample that is at least one of processed by a processing system and to be processed by a processing system, the processing system having a controller configured for managing aspects for processing workpieces with the processing system in response to an analysis of the workpieces separate from the QA analysis, the method including: capturing QA image sensor data of a QA sample with an image sensor assembly of a QA system; generating, with a computing device, at least one of a 2D model and a 3D model of the QA sample using the QA image sensor data; and performing, with a computing device, a QA analysis of the QA sample by comparing QA data of the at least one of the 2D model and 3D model of the QA sample with a QA specification of the QA sample.

In some aspects, the techniques described herein relate to a quality assurance (QA) system for performing a QA analysis of a QA sample that is at least one of processed by a processing system and to be processed by a processing system, the processing system having a controller configured for managing aspects for processing workpieces with the processing system in response to an analysis of the workpieces separate from the QA analysis, including: an image sensor assembly of a QA system configured to capture QA image sensor data of a QA sample; a processor; and a memory storing instructions that, when executed by the processor, cause a computing device of the QA system to: generate at least one of a 2D model and a 3D model of the QA sample; and perform a QA analysis of the QA sample by comparing QA data of the at least one of the 2D model and 3D model of the QA sample and a QA specification of the QA sample.

In some aspects, the techniques described herein relate to a computer-implemented method of performing a quality assurance (QA) analysis for a QA sample that is at least one of processed by a processing system and to be processed by a processing system, the processing system having a controller configured for managing aspects for processing workpieces with the processing system in response to an analysis of the workpieces separate from the QA analysis, the method including: capturing QA image sensor data of a QA sample with an image sensor assembly of a QA system; generating, with a computing device, at least one of a 2D model and a 3D model of the QA sample using the QA image sensor data; obtaining a QA weight measurement of the QA sample with a weight measurement assembly of the QA system; and performing, with a computing device, a QA analysis of the QA sample, including: comparing QA image data of the at least one of the 2D model and 3D model of the QA sample with a QA specification of the QA sample; and comparing at least one of the QA weight measurement and a calculated density of the QA sample based on the QA weight measurement with at least one of weight and density values determined from at least one of a QA specification of the QA sample and a scan of the QA sample by the processing system.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1 shows a block diagram of a non-limiting example of a food processing management system according to various aspects of the present disclosure.

FIG. 2 shows a schematic illustration of a non-limiting example of a processing system according to various aspects of the present disclosure.

FIG. 3 is a block diagram of a non-limiting example of a processor computing device according to various aspects of the present disclosure.

FIGS. 4A-4D are views of an example of a QA scanning station according to various aspects of the present disclosure.

FIG. 5A is a top isometric view of an alternative example of a QA scanning station according to various aspects of the present disclosure.

FIG. 5B is a bottom isometric view of the QA scanning station of FIG. 5A.

FIG. 6 is an isometric view of an alternative example of a QA scanning station according to various aspects of the present disclosure.

FIG. 7 is a block diagram of a non-limiting example of a quality assurance (QA) computing device according to various aspects of the present disclosure.

FIG. 8 a flowchart that illustrates a non-limiting example of a method of performing a QA analysis for a workpiece.

FIG. 9 is a block diagram that illustrates a non-limiting example of a computing device appropriate for use as a computing device with examples of the present disclosure.

### DETAILED DESCRIPTION

Aspects of the present disclosure are directed to systems and methods for ensuring the quality of processed workpieces, such as food products and machine components.

Food processing lines are generally monitored by quality assurance (QA) personnel to ensure that processed products and machine components are within specifications (“spec”).

With regard to food products, QA personnel will often take a small sample of the products being processed to ensure that they are being portioned, cut, trimmed, etc., according to the required specs. For instance, a QA technician may take a sample of ten portioned/trimmed chicken breasts (out of thousands of incoming breasts or chicken cuts) and measure and/or weigh the chicken breast to determine if the chicken breast or breast portion is the correct size, weight, shape, etc.

Measurements to assess product size may include taking one measurement each for height, length, and width. The QA technician may also overlay the sample on a laminated sheet showing the required product outline to assess whether the sample is the correct shape. Further, the QA technician may weigh the sample on a bench scale to acquire an accurate weight of the sample to determine if the sample is the correct weight. Other aspects may also be assessed depending on the product specifications. For instance, certain food suppliers may require that their beef patty have a certain “bun coverage” area or a certain shape. Other suppliers may require no blood spots or bruises on the portioned meat.

While QA technicians are often highly skilled, there will always be variation between technicians, and over time manual measurements are prone to inaccuracies and inconsistencies. Additionally, there can be wide variation in skills and experience between technicians, leading to variation in data. In that regard, the QA measurements are not standardized across food processing lines or across production runs.

Moreover, only a small number of food product samples may be measured given the amount of time it takes to obtain the measurements. In that regard, the data pertaining to the samples may not accurately represent all the processed products for the production run. Nevertheless, without the ability to measure every processed food product, if a threshold level of samples (e.g., more than 5%) are out of spec, a production manager may rework all of the food products (e.g., re-run the entire food product through the machine), downgrade the food products, and/or discard the entire run.

When taking the measurements, the QA technician may reference a spec sheet to determine whether the food product is in spec or out of spec. However, the QA technician often does not have the time or capacity to conduct any further analysis, such how far out of spec the food product is (e.g., the percentage out of spec), what adjustments can be made to the machine to bring the food product into spec, etc. Instead, the technician will often make guesstimated, on the fly adjustments to the machine to try to account for the out of spec processing. For instance, the technician may adjust a density value of an incoming food product to change the portioned size, weight, shape, etc.

In that regard, even when the measurements are accurately taken, the data is not necessarily used in an efficient and effective manner to improve the quality of the processed product. For instance, the data must be manually entered into a computing device if any analysis is desired. Moreover, any analysis would be after the fact, when a significant portion of (or all of) the incoming product has already been processed.

As noted above, food processing machines typically use various scanning techniques to ascertain a size, shape, count, etc., of a food product as it is being advanced on a moving conveyor. This information is analyzed with the aid of a computer to determine, for instance, how to most efficiently and accurately portion the food product into optimum sizes, how to trim the product (e.g., locating the fat for trimming), how to harvest the product (e.g., sort and/or pickup products of various sizes for further processing or packaging), etc.

With regard to machine components, it is often difficult to proactively conduct a QA analysis because the components are part of a complex system. In that regard, high level aspects of a machine may be monitored, such as belt speeds, oven temperatures, etc. However, it may be beneficial to monitor or perform QA of individual machine components, such as conveyance system components (e.g., pins, belt pickets or rods, links, chains, mesh components, etc.), cutting components, or other high wear components. Proactively addressing machine component issues may be used to assess belt sag, belt wear, blade wear, or other issues before they affect processing accuracy.

Examples of the present disclosure are directed to quality assurance (QA) systems and methods that may be used to assess attributes of a workpiece, such as characteristics of a workpiece before it is processed by a processing machine and/or the quality of a workpiece after it is processed. In some examples, the exemplary QA systems and methods disclosed herein may be used to obtain accurate dimensions of a processed workpiece, such as by using data from one or more sensors configured to capture image data of the processed workpiece. In some examples, the exemplary QA systems and methods disclosed herein may be used to obtain accurate weight measurements of a processed workpiece, and the weight measurements may be obtained substantially simultaneously with any image data or other data.

Image data, weight data, and any other data pertaining to a workpiece (“QA data”) that is gathered by the QA system (such as machine learning model output data, as discussed below) may be processed by a computing device(s) of the QA system for assessing one or more attributes of a workpiece. For example, weight measurements may be used with dimensional data to determine additional parameters of a processed workpiece, such as its density. By knowing the density value of a workpiece (or a plurality of workpieces), density settings in the processing machine may be automatically adjusted for accurately portioning the remaining workpieces. In that regard, the QA systems and methods disclosed herein may be used to determine the density of a workpiece(s) before it is processed by a processing machine such that the density settings in the machine may be automatically adjusted for accurately portioning the incoming workpiece(s). In other aspects, the QA systems and methods disclosed herein may be used to determine the density of a workpiece(s) after it is processed by a processing machine such that the density settings in the machine may be automatically adjusted for accurately portioning remaining workpiece(s).

QA data may be processed by the QA system or another computing device to generate additional data for use in assessing a workpiece or operational aspects pertaining to a workpiece. For instance, in some examples, the QA systems and methods disclosed herein may be used to simply determine whether a workpiece is in spec or out of spec and indicate such to QA personnel. In some examples, when a workpiece is out of spec, the QA systems and methods disclosed herein may be used to determine how far out of spec the workpiece is and provide a list of possible corrective actions that may be taken by QA personnel and/or automatically change a setting in the machine (or automatically order or suggest ordering of a replacement part or repair of the part in the case of a machine component).

In some examples, the QA systems and methods disclosed herein may be used to assess a production run, such as by sending QA data to a monitoring system for further processing. In some examples, the QA systems and methods disclosed herein may be used to identify one or more additional processing steps for the workpiece, such as trimming, tenderizing, sorting, picking, packaging, etc. In some examples, the QA systems and methods disclosed herein may be used to perform a global optimization to assign the processed workpiece to a package configuration based on the assessment.

As will become appreciated, the QA systems and methods disclosed herein allow a user to obtain significantly higher accuracy QA data, such as compared to data obtained from manual measurements taken with calipers and a scale. Further, the QA data obtained using the QA systems and methods disclosed herein can be obtained in a fraction of the time it takes to perform manual measurements. In that regard, the QA systems and methods disclosed herein can be used to obtain highly accurate QA data in less time compared to prior art methods.

In addition, the QA systems and methods disclosed herein are configured to directly process and use the QA data without the need for any manual inputs, reducing or eliminating data transmission errors. The QA data and any related data can be processed to determine appropriate machine adjustments or part repair/replacement, which may be carried out manually or automatically.

In that regard, the QA systems and methods disclosed herein can be used to automatically record and store data for historical review. At least one of short and long term historical data may be used in controlling automation of a processing apparatus (e.g., automatically change a setting in the machine, automatically order or suggest ordering of a replacement part or repair of the part, etc. For instance, a short term running average of historical QA data may be used for control/automation, rather than using an instantaneous QA value. In other aspects, artificial intelligence, such as one or more machine learning models may use short and/or long term historical QA data for baseline data and training one or more machine learning models.

In that regard, the QA systems and methods disclosed herein can be used to continually train and update aspects of the QA systems and methods during routine monitoring. For instance, the QA data generated and processed by the QA systems and methods disclosed herein may be used to train one or more machine learning models, which can be used to perform at least one of QA analysis, provide a list of possible corrective actions that may be taken by QA personnel, automatically change a setting in the machine, automatically order or suggest ordering of a replacement part or repair of the part in the case of a machine component, etc.

The QA systems and methods disclosed herein provide a comprehensive approach that assists the QA personnel in the routine monitoring of production by providing faster, more accurate identification of defects or specification levels with the ability to continually train and update the system during routine monitoring. The foregoing benefits as well as other benefits will be further appreciated from the description that follows.

In the present disclosure, references to “food,” “food products,” “food pieces,” “food items,” “pieces,” “portions,” etc., are used interchangeably and are meant to include all manner of foods. Such foods may include meat, fish, poultry, plant-based products, fruits, vegetables, nuts, or other types of foods. Also, the QA systems and methods are directed to raw food products, as well as partially and/or fully processed or cooked food products.

Further, the exemplary QA systems and methods disclosed herein, though sometimes described with specific applicability to food products or food items, may also be used outside of the food area. For instance, the exemplary QA systems and methods disclosed herein may be applicable to machine components or other workpieces. Accordingly, the present disclosure may reference “workpieces,” “products”, “components”, “samples”, etc., which terms are synonymous with each other. It is to be understood that references to “workpieces,” “products”, “components”, “samples”, etc., also include food, food products, food pieces, food items, etc. Moreover, references to “food,” “food products,” “food pieces,” “food items,” “pieces,” “portions,” etc., also include “workpieces,” products, components, samples, etc.

Moreover, “QA sample” may be used to generally refer to any workpiece, food product, etc., that is analyzed using the QA systems and methods disclosed herein. In that regard, a QA sample may include a workpiece that has already been at least partially processed by a processing system, an incoming workpiece that has not yet been processed, a used component, a new component, etc. Moreover, when referring to a “workpiece” or the like, it may also include a QA sample.

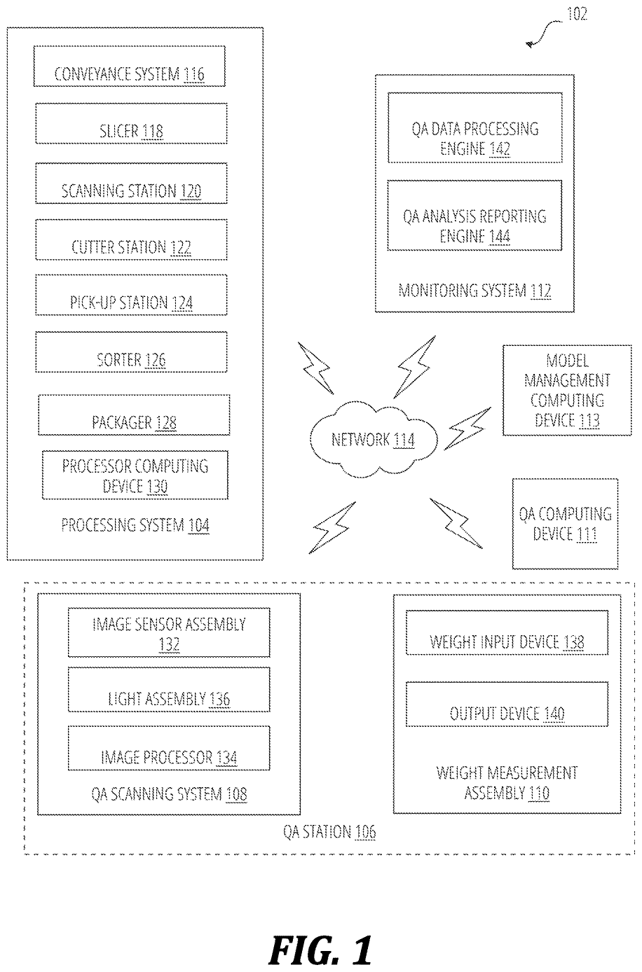

FIG. 1 depicts a schematic illustration of a non-limiting example of a workpiece processing management system 102 that can be used to gather and process QA data for assessing one or more attributes of a workpiece processed by a workpiece processing machine or system and/or to be processed by the machine or system (or a “QA sample”). The workpiece processing management system 102 may include various networked computing devices configured for carrying out aspects of gathering and processing QA data for assessing one or more attributes of the QA sample, as well as other aspects of processing the QA sample and/or workpieces of the same type (e.g., conveying, slicing, portioning, sorting, packaging, etc.).

In the depicted example, the workpiece processing management system 102 includes a processing system 104, a QA station 106 having an integrated QA scanning system 108 and weight measurement assembly 110, a QA computing device 111, an optional monitoring system 112, and a model management computing device 113 communicatively coupled together through a network 114. The network 114 can be any kind of network capable of enabling communication between the various components of the workpiece processing management system 102. For example, the network can be a WiFi network.

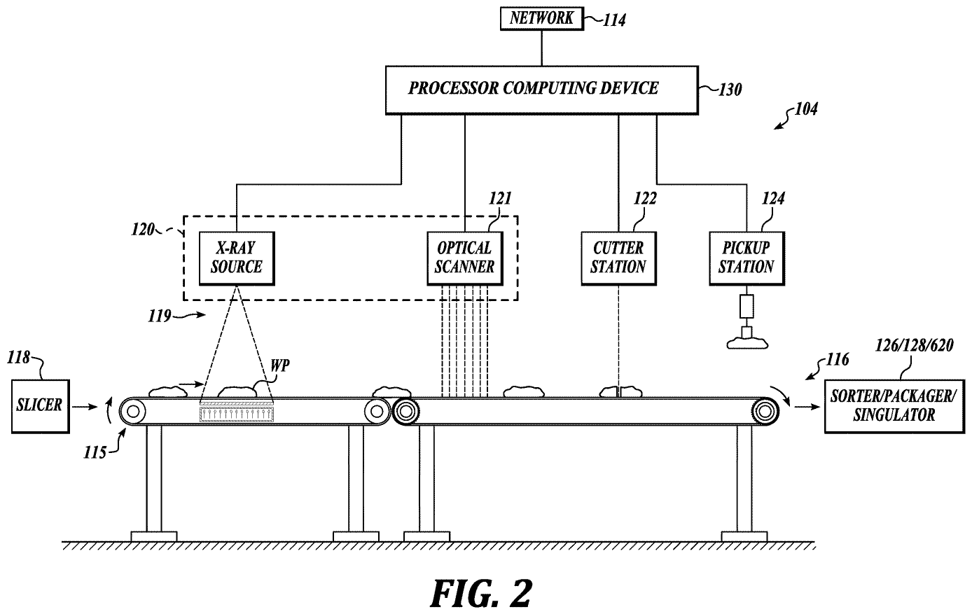

The processing system 104 will first be described with reference to FIGS. 1 and 2. The processing system 104 is generally configured to carry out processing of the workpiece before the workpiece is designated as a QA sample to be scanned and/or weighed by the QA station 106. In that manner, the quality of workpieces processed by the processing system 104 (such as whether the workpieces are in spec) may be analyzed with data gathered from the QA station 106. However, in some examples, a QA station 106, including a QA scanning system 108 and/or a weight measurement assembly 110 may additionally or alternatively be located upstream of the processing system 104 for gathering QA data relevant to the incoming workpieces.

The processing system 104 includes a conveyance system 116 or another movement device configured to carry workpieces WP, or workpieces between various portions of the processing system 104. For instance, the conveyance system 116 may carry the workpieces between one or more of a slicer 118, a scanning station 120, a cutter station 122, a pick-up station 124, a sorter 126, and a packager 128. The various components of the processing system 104 may be controlled by a processor computing device 130.

The conveyance system 116 may include a powered belt 115 which is supported by a series of rollers (not labeled), one of which is the drive roller, which drives the belt 115 in a standard manner. An encoder may be employed with respect to a support roller or an end roller to determine the position of the workpiece on the conveyor belt as well as the progress or movement of the workpiece in the conveyance direction. Although a single belt 115 is shown, the conveyance system 116 may be composed of one or more belts, for instance, a flat, solid belt may support the workpiece during scanning under a portion of scanning station 120. Such belts are typically flat, non-metallic belts. The workpiece can be transferred from the first belt to the second belt which supports the workpiece during the portioning or trimming process at cutter station 122. If a waterjet cutter is used to portion or trim the workpiece, it is advantageous to utilize an open mesh, metallic belt to allow the waterjet to pass downwardly therethrough, and also so that the belt is of sufficient structural integrity to withstand the impact thereon from the waterjet. Such metallic, open mesh belts are articles of commerce.

Slicer 118 may be used to slice a primal product (e.g., a cut of meat initially separated from the carcass of an animal during butchering or processing, such as a pork loin) into a sub-primal product (such as a sirloin chop, center loin chop, center rib chop, and rib end chop for a pork loin) before being further processing by processing system 104. In that regard, the slicer 118 may be located downstream from a cutter (not shown) used to cut a carcass into primal cuts. The slicer 118 may also be used to cut a sub-primal cut, such as a pork chop or a chicken breast, into slices.

Various types of slicers may be utilized to slice the workpiece into one or more desired thicknesses of cuts or slices. The slicer 118 may be configured to cut through muscle and optionally bone, and it may be oriented vertically or horizontally. For example, the slicer 118 may be in the form of a high-speed water jet, a laser, a rotary saw, a hacksaw, or band saw.

The slicer 118 may be adjustable so that a desired thickness of each cut or slice is obtained. Such adjustment may be under the control of a processor, such as the processor computing device 130. For example, the slicer 118 may be adjusted based on data sent from the QA scanning system 108 and processed by the processor computing device 130 and/or the QA computing device 111, such as to account for different density values of the workpiece (e.g., if a higher density is measured by the QA scanning system 108, a smaller slice may be made to achieve a slice within a weight spec). In examples where the slicer 118 is oriented to slice horizontally, the thickness of each cut or slice required to produce the desired target weight may be dependent on any workpiece undercuts, voids, or other irregularities. In that case, measured QA data of sliced QA samples may be used to adjust slicer settings (e.g., a horizontal slicing height offset) to account for any workpiece irregularities. In some examples, the processing system 104 receives cut or sliced products from another machine or location, and the slicer 118 is excluded. Generally, the terms “slicing”, “portioning”, “cutting”, ‘trimming”, or the like may include any type of, or any combination of, product cutting (e.g., slicing alone, portioning alone, or any other type of product cutting, and any combination of slicing, portioning, and other type of product cutting).

The workpieces WP are inspected at scanning station 120 to ascertain physical parameters or characteristics of the workpieces, pertaining to, for example, size and/or shape of the workpieces. Such characteristics may include, for example, the length, width, length/width aspect ratio, thickness, thickness profile, contour, outer contour configuration, outer taper, flatness, outer perimeter configuration, outer perimeter size and shape, volume, weight, as well as whether the workpieces contain any undesirable materials, such as bones, fat, cartilage, metal, glass, plastic, etc., and the location of the undesirable materials in the workpieces.

Such physical parameters may include the maximum, average, mean, and/or medium values of such parameters. With respect to the thickness profile of the workpiece, such profile can be along the length of the workpiece, across the width of the workpiece, as well as both across/along the width and length of the workpiece.

The parameter referred to as the “perimeter” of the workpiece refers to the boundary or distance around a workpiece. Thus, the terms outer perimeter, outer perimeter configuration, outer perimeter size, and outer perimeter shape pertain to the distance around the configuration, the size and the shape of the outermost boundary or edge of the workpiece, etc.

The foregoing enumerated size and/or shape parameters/characteristics are not intended to be limiting or inclusive. Other size and/or shape parameters/characteristics may be ascertained, monitored, measured, etc., by the systems and methods disclosed herein. Moreover, the definitions or explanations of the above specific size and/or shape parameters/characteristics discussed above are not meant to be limiting or inclusive.

The scanning station 120 may include any suitable scanners, such as one or more of the scanners and/or systems and methods for processing scanner data described in U.S. Pat. No. 10,721,947, entitled “Apparatus for acquiring and analy sing product-specific data for products of the food processing industry as well as a system comprising such an apparatus and a method for processing products of the food processing industry,” hereby incorporated by reference herein in its entirety.

In the depicted example, the scanning station 120 may utilize an x-ray apparatus 119 for determining the physical characteristics of the workpiece, including its shape, mass, and weight. X-rays may be passed through the object in the direction of an x-ray detector (not labeled). Such x-rays are attenuated by the workpiece in proportion to the mass thereof. The x-ray detector is capable of measuring the intensity of the x-rays received thereby, after passing through the workpiece. This information may be utilized to determine physical parameters pertaining to the size and/or shape of the workpiece, including for example, the length, width, aspect ratio, thickness, thickness profile, contour, outer contour configuration, perimeter, outer perimeter configuration, outer perimeter size and/or shape, volume, weight, as well as other aspects of the physical parameters/characteristics of the workpiece. With respect to the outer perimeter configuration of the workpiece, the X-ray detector can determine locations along the outer perimeter of the workpiece based on an X-Y coordinate system or other coordinate system. An example of such x ray scanning devices are disclosed in U.S. Pat. No. 5,585,605, entitled “Optical-scanning system employing laser and laser safety control”, U.S. Pat. No. 10,654,185, entitled “Cutting/portioning using combined X-ray and optical scanning”, U.S. Pat. No. 5,585,603, entitled “Method and system for weighing objects using X-rays”, as well as U.S. Pat. No. 10,721,947 (referenced above), incorporated herein by reference in their entirety.

The scanning station 120 may also include an optical scanner 121 for generating at least one of a visible light (e.g., greyscale) image, a laser light scattering image, a height map, a hyperspectral image, a multispectral image, etc., of the workpiece to show one or more of the overall shape/size of the workpiece, a composition of the workpiece (e.g., fat. v. lean meat), a height or thickness over the area of the workpiece, etc. Scanning with the scanning station 120 can be carried out using a variety of techniques, such as the techniques shown and described in U.S. Pat. Nos. 10,654,185, 10,721,947, and 11,570,998, all incorporated by reference in their entirety.

The optical scanner 121 may include a video camera (not shown) to view a workpiece illuminated by one or more light sources. Light from the light source is extended across the moving conveyor belt 115 to define a sharp shadow or light stripe line, with the area forwardly of the transverse beam being dark. When no workpiece is being carried by the conveyor belt 115, the shadow line/light stripe forms a straight line across the belt. However, when a workpiece passes across the shadow line/light stripe, the upper, irregular surface of the workpiece produces an irregular shadow line/light stripe as viewed by a video camera (not shown) directed diagonally downwardly on the workpiece and the shadow line/light stripe. The video camera detects the displacement of the shadow line/light stripe from the position it would occupy if no workpiece were present on the conveyor belt. This displacement represents the thickness of the workpiece along the shadow line/light stripe.

The length of the workpiece is determined by the distance of the belt travel that shadow line/light stripes are created by the workpiece. In this regard, the encoder, integrated into the conveyor 115, generates pulses at fixed distance intervals corresponding to the forward movement of the conveyor.

In some examples, the scanning station 120 uses a single SICK® camera with a single laser light source that is suitable for capturing optical data and generating two or more images/views based on the optical data. For instance, the single camera may be in communication with a separate processor (having one or more feature recognition modules or the like) and/or the processor computing device 130 for generating one or more views from the captured optical data, such as a fat recognition (FRS) object view, a laser scatter object view, and a height mode object view.

In some examples at least two optical cameras optionally each equipped with a different imaging processor are used. For example, a simple optical camera, for example a greyscale camera, and/or RGB camera and/or IR and/or UV camera and/or a charge coupled device (CCD), can be used to acquire and/or generate one or more complete images of the workpiece for detecting certain characteristics, such as, e.g., the outer contour of the workpiece. Moreover, a second, special camera, for example a multispectral or hyperspectral camera, can be used to acquire images/data of specific regions or characteristics of the workpiece, such as blood spots, streaks of fat or the like. It should be appreciated that a single camera/scanner may instead be used to capture all the data needed to generate the various images, such as with various imaging processes.

The results of the scanning occurring at scanning station 120 are transmitted to the processor computing device 130.

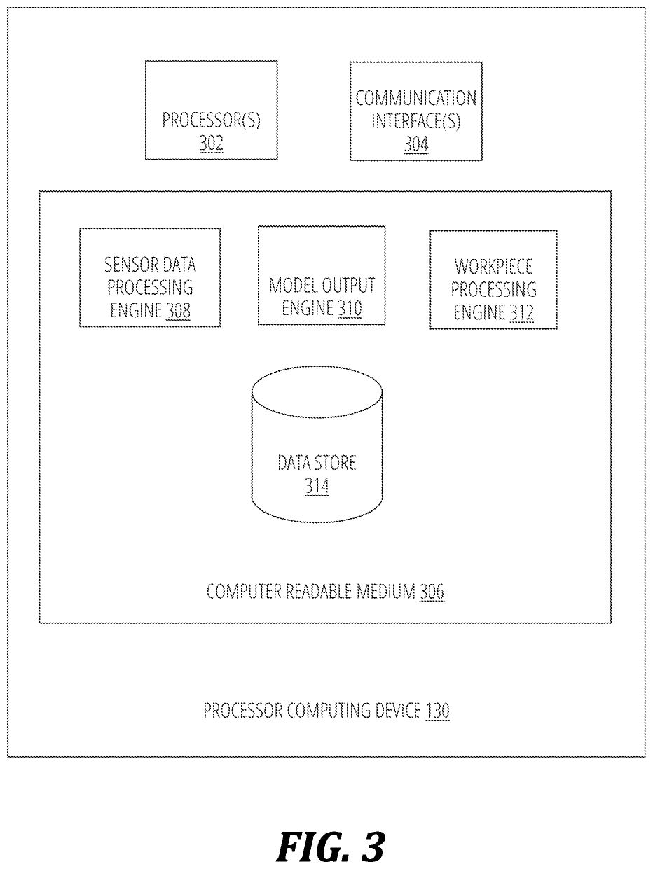

Exemplary aspects of the processor computing device 130 will now be described with reference to FIG. 3. As noted above, the processor computing device 130 may be generally configured for controlling components of the processing system 104, such as in response to scan data transmitted from the scanning station 120 and other data or inputs (such as from the QA computing device 111). In the exemplary block diagram of FIG. 3, the processor computing device 130 includes a processor(s) 302, a communication interface(s) 304, computer readable medium 306, and at least one data store 316. As shown, the computer readable medium 306 has stored thereon logic that, in response to execution by the one or more processor(s) 302, cause the processor computing device 130 to provide a sensor data processing engine 308, a model generation engine 310, and a workpiece processing engine 312.

The processor computing device 130 may be implemented by any computing device or collection of computing devices, including but not limited to a desktop computing device, a laptop computing device, a mobile computing device, an edge computing device, a programmable logic controller (PLC), a server computing device, a computing device of a cloud computing system, and/or combinations thereof. In some examples, the processor(s) 302 may include any suitable type of general-purpose computer processor. In some examples, the processor(s) 302 may include one or more special-purpose computer processors or AI accelerators optimized for specific computing tasks, including but not limited to graphical processing units (GPUs), vision processing units (VPTs), and tensor processing units (TPUs).

In some examples, the communication interface(s) 304 includes one or more hardware and or software interfaces suitable for providing communication links between components. The communication interface(s) 304 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof.

As used herein, “computer-readable medium” refers to a removable or nonremovable device that implements any technology capable of storing information in a volatile or non-volatile manner to be read by a processor of a computing device, including but not limited to: a hard drive; a flash memory; a solid state drive; random-access memory (RAM); read-only memory (ROM); a CD-ROM, a DVD, or other disk storage; a magnetic cassette; a magnetic tape; and a magnetic disk storage.

As used herein, “engine” refers to logic embodied in hardware or software instructions, which can be written in one or more programming languages, including but not limited to C, C++, C#, COBOL, JAVA™, PHP, Perl, HTML, CSS, Javascript, VBScript, ASPX, Go, and Python. An engine may be compiled into executable programs or written in interpreted programming languages. Software engines may be callable from other engines or from themselves. Generally, the engines described herein refer to logical modules that can be merged with other engines or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device.

As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

The sensor data processing engine 308 of the processor computing device 130 may be configured to process incoming sensor data for a workpiece. The sensor data may include one or more images captured by the scanning station 120. For instance, the sensor data may include one or more images generated by the x-ray apparatus 119 and the optical scanner 121.

The sensor data processing engine 308 may be configured to execute one or more feature recognition modules for generating views/images from the scan data and/or processing data from the different views. For instance, the sensor data processing engine 308 may be configured to generate at least one of a fat recognition (FRS) object view, a laser scatter object view, and a height mode object view, such as from data captured with the optical scanner 121.

Before executing one or more feature recognition modules, the sensor data processing engine 308 may first analyze the data from the X-ray apparatus 119 and the optical scanner 121 to confirm that the workpiece scanned by the optical scanner 121 is the same as the workpiece previously scanned by X-ray apparatus 119 and/or whether the workpiece has moved or shifted during transfer between conveyors, as discussed in U.S. Pat. Nos. 10,654,185 and 10,721,947 (referenced above), incorporated by reference herein. In that regard, a comparison of the X-ray and optical data may be processed by the processor computing device 130.

The sensor data processing engine 308 may be configured to generate a registered scan of a workpiece including a first scan of a first scan type (e.g., x-ray) and a second scan of a second scan type (e.g., an optical image). For instance, a registered scan of the workpiece may be generated by the sensor data pre-processing engine 308, which maps an X-ray image of the workpiece scanned at the x-ray apparatus 119 onto a (possibly transformed) optical image of the workpiece as scanned by optical scanner 121. In one example, the registered scan is generated by the sensor data pre-processing engine 308 using the systems and methods described in U.S. Pat. No. 10,654,185, incorporated herein by reference in its entirety. For instance, the X-ray data may be mapped onto the optical data, optionally with a transformation or translation of one or more of the images/data to account for any movement/shifting of the workpiece on a conveyor.

Data processed by the sensor data processing engine 308 may be transmitted to or retrieved by the model generation engine 310 for generating one or more of a 2D and 3D model of the scanned workpiece. The model generation engine 310 may include software modules suitable for processing scan data and generating 3D models (showing contour, shape, volume, texture, etc.), 2D models (e.g., showing a height and outline), or other images. For instance, the model generation engine 310 may run the proprietary DSI Q-LINK™ Portioning Software developed by Design Systems, Inc. of Redmond, Washington

The model generation engine 310 may execute one or more feature recognition modules for generating 2D views/images/models from the scan data. The one or more feature recognition modules may be executed by the model generation engine 310 in addition to or instead of execution by the sensor data processing engine 308. For instance, the model generation engine 310 may be configured to generate at least one of a fat recognition (FRS) object view, a laser scatter object view, and a height mode object view. A 2D model of a workpiece may be used to identify a contour of a workpiece, shape irregularities of the workpiece, height, etc.

The model generation engine 310 may also or instead be configured to generate 3D models of a scanned workpiece. A 3D model of the workpiece may be used to determine how to cut the workpiece into desired portions and/or trim the workpiece into a desired overall shape.

The cutting, portioning, trimming, etc., of a workpiece may be carried out by the workpiece processing engine 312. The workpiece processing engine 312 may analyze data received from the sensor data processing engine 308 and/or the model generation engine 310 to determine cutting paths for the cutter station 122 as well as other processing steps (e.g., sorting, picking, harvesting, etc.). The workpiece processing engine 312 may also receive/process instructions from the QA computing device 111 to make any necessary cutting or processing adjustments to ensure subsequently processed workpieces are within the required spec, to optimize use of processed workpieces, etc.

For instance, if the workpiece processing engine 312 receives instructions from the QA computing device 111 regarding a process adjustment, the processing system 104 may carry out further scanning at scanning station 120 and analyze that scanning data to determine how to cut, portion, trim, or otherwise process at least some of the remaining or unprocessed workpieces for that production run. In some examples, if the workpiece processing engine 312 receives instructions from the QA computing device 111, the workpiece processing engine 312 may be able to carry out all necessary cutting or other processing of the workpiece without conducting any further scanning or analysis.

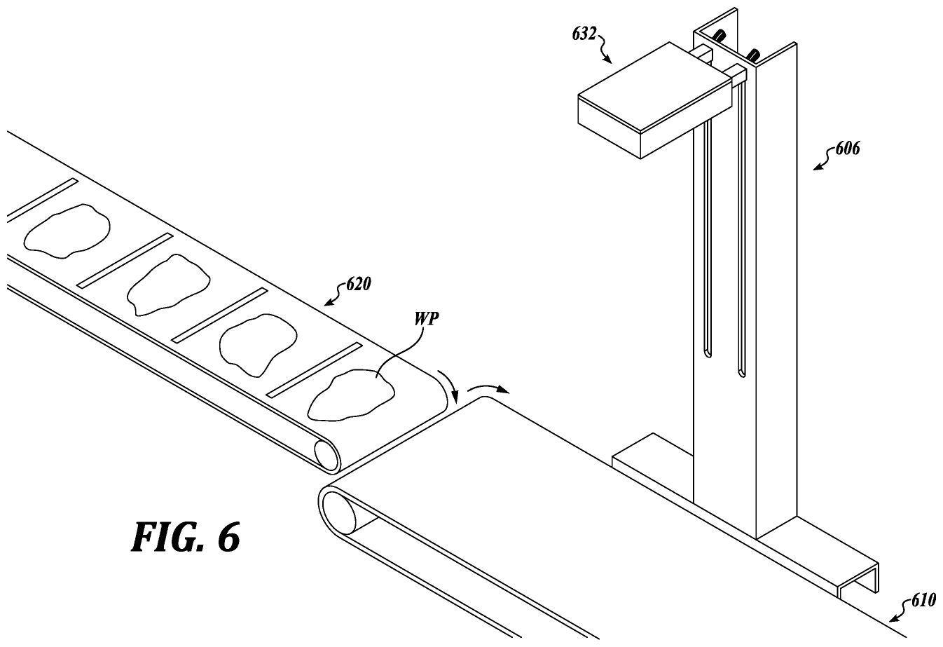

Returning to FIG. 2, after any processing (e.g., cutting, portioning, trimming, etc.), the workpiece (and/or any material removed from the workpiece) may be transferred to a takeaway conveyor (such as a singulator of the conveyance system 116, as shown in FIG. 6), a storage bin, the sorter 126, the packager 128, or other location, such as with a pick-up station 124. The pick-up station 124, sorter 126, and packager 128 may receive instructions from the processor computing device 130 based upon the processed scan data from scanning station 120.

For example, if the workpiece is portioned into pieces, the processor computing device 130 may instruct the pick-up station 124 and/or the sorter 126 to remove or divert trim pieces or other unwanted pieces from the conveyor. Trim pieces may be removed or diverted based on, for instance, their known location on the conveyor resulting from the cutting instructions, scan data from the scanning station 120 indicating that the incoming product was not the correct shape/size/type to produce certain portions, QA data indicating that the portioned pieces are out of spec, etc. In another example, the processor computing device 130 may instruct the pick-up station 124 and/or the sorter 126 to transfer all portions of a certain type to a designated conveyor, bin, etc., for packaging together, such as based on information received by the QA computing device 111.

Although FIGS. 1, 2, and 3 depict specific components and sub-assemblies of a processing system, it should be appreciated that any other suitable arrangement of processing components may be used. For instance, the processing system 104 may incorporate aspects of the systems shown and described in U.S. Pat. No. 7,651,388, entitled “Portioning apparatus and method”, U.S. Pat. No. 7,672,752, entitled “Sorting workpieces to be portioned into various end products to optimally meet overall production goals”, and U.S. Pat. No. 8,688,267, entitled “Classifying workpieces to be portioned into various end products to optimally meet overall production goals”, hereby incorporated by reference herein in their entirety.

Referring back to FIG. 1, exemplary aspects of the QA station 106 will now be described. As noted above, the QA station 106 is configured to gather and process QA data for assessing one or more attributes of a QA sample processed by the processing system 104 or to be processed. The QA station 106 may include an integrated QA scanning system 108 and weight measurement assembly 110. The QA computing device 111, although shown separately, may also define an integral part of the QA station 106. In general, the QA station 106 may be configured to capture, package, and send QA image data, QA weight data, and any other relevant data (“QA data”) for a QA sample to a computing device (such as the QA computing device 111, the monitoring system 112, the model management computing device 113, and/or the processor computing device 130), for performing a QA analysis or otherwise processing the QA data. The QA analysis may include determining whether the QA sample is within spec and optionally determining any corresponding steps that should be taken.

Exemplary aspects of the QA scanning system 108 will first be described.

FIG. 1 depicts a block diagram that illustrates aspects of a non-limiting example of the QA scanning system 108 according to various aspects of the present disclosure. In the depicted example, the QA scanning system 108 includes an image sensor assembly 132 having at least one image sensor for capturing QA image data of the QA sample being viewed. The QA scanning system 108 may further include a light assembly 136 configured to sufficiently illuminate the QA sample being captured by the image sensor assembly 132. The QA scanning system 108 may further include an image processor 134, which may be used to receive, process, and package QA image data captured with the image sensor assembly 132 for sending to the QA computing device 111 or another computing device (such as the processor computing device 130 of the processing system 104). The QA scanning system 108 may include any other components necessary or suitable for the application and/or environment, such as a heater, a conveyance system, etc. Moreover, the QA scanning system 108 used in the systems and methods described herein excludes any type of scanning that could be done by human observation, which would not support the needed processing speed and accuracy of the workpiece processing management system 102.

The image sensor assembly 132 of the QA scanning system 108 will first be described in detail. The image sensor assembly 132 may include any suitable image sensor or combination of image sensors for capturing and/or generating QA image data relevant for assessing the quality of the intended QA sample. In some examples, the image sensor assembly 132 may include one or more cameras for capturing still images of a QA sample and a support surface on which it rests. For instance, the light assembly 136 may include one or more optical still cameras, for example a greyscale camera, an RGB camera, an infrared (IR) and/or UV camera, thermal imaging device, thermal camera, a charge coupled device (CCD), etc. An optical still camera can be used to acquire and/or generate one or more complete still images of a QA sample for detecting certain characteristics, such as, e.g., the outer contour or perimeter of the QA sample, a depth or height of the QA sample, etc. Such QR still image data showing the outer contour of the QA sample may be used to determine if a product meets the shape and/or size spec for the product, such as a chicken breast having a required length/width and/or a certain bun coverage.

The image sensor assembly 132 may also include one or more additional still cameras, for example a multispectral or hyperspectral camera. Additional still cameras such as multispectral or hyperspectral cameras can be used to acquire images/data of specific regions or characteristics of a QA sample, such as blood spots, streaks of fat, woody chicken striations, or the like.

In some examples, the image sensor assembly 132 may be movable relative to a stationary or fixed QA sample. For instance, in one example, the image sensor assembly 132 may include one or more image sensors placed at the end of a robot arm such that numerous still images may be taken of a QA sample and/or its surroundings with the image sensor from various angles. In such an example, the image sensor assembly 132 may include one or more still cameras or other cameras (e.g., a stereo camera). In such an example, the robot-controlled image sensor assembly 132 may capture enough images of a QA sample to generate a 3D model of the QA sample. The support surface on which a QA sample is placed may also be captured by the image sensor for determining the sensor's distance from the surface, which may be used in calculating the product height. The image sensor assembly 132 may also or instead include one or more image sensors mounted to a linear actuator, as described below.

It can be appreciated that capturing images at various angles can generate even more reliable, complete image data of a QA sample. In other words, using a still camera or other camera moveable relative to the imaging support surface allows for viewing of a QA sample at more/better angles, thereby eliminating QA image data gaps and improving QA image accuracy.

In the examples described herein, the image sensor assembly 132 may also or instead include image sensor technology suitable for capturing image data needed to generate a 3D model of a QA sample and/or a 2D representation of the height or elevation of the scene. In some examples, the image sensor assembly 132 includes at least one of a 3D vision system or 3D laser scanning technology like LiDAR (Light Detection and Ranging), structured light scanning, or photogrammetry, a stereo depth camera, a Time-of-Flight (ToF) stereoscopic camera, or the like, or combinations thereof.

In some examples, the image sensor assembly 132 includes a stereo depth (e.g., stereoscopic) camera configured to generate a 3D depth image or height map of the QA sample. For instance, at least one Intel RealSense Depth Camera (e.g., D405) may be used. When using one or more stereo depth cameras or in a similar image sensor assembly configuration, each camera may be configured to optimize the contrast of captured images (e.g., minimizing white pixels) to optimize object segmentation, e.g. separation of the QA sample from the background and miscellaneous noise within the image. In that regard, each camera may also be configured to optimize resolution, exposures, frame rate, etc., to maximize an accuracy of a still camera image. In some examples, multiple still images of the same QA sample are acquired to define a QA image, and a temporal filter may be used to filter out temporal noise in the image.

In one example, the image sensor assembly 132 includes a structured light source and scanner configured to capture QA sample depth and surface information for generating a height map or 3D model of the product and/or a 2D representation of the height or elevation of the scene (sometimes also referred to herein as a “3D laser scanner” or the like). In one example, the structured light source/scanner is a SICK® TriSpector1000 3Dlaser scanner. A 3D laser scanner such as the SICK® TriSpector 1000 3Dlaser scanner digitally captures the size and shape of physical objects using a line of laser light. Generally, with a 3D laser scanner, a laser probe projects a line of laser light onto a surface supporting an object to be scanned while sensor cameras continuously record the changing distance and shape of the laser line in three dimensions (XYZ) as it sweeps along the object. The shape of the object appears as millions of points called a “point cloud” on the computer monitor as the laser moves around capturing the entire surface shape of the object.

For the laser to sweep along a QA sample, the structured light scanner may be movable relative to an imaging support surface on which the QA sample rests, and/or the imaging support surface may be movable relative to the structured light scanner. For instance, the structured light scanner may be movable by a robot or an actuator relative to the imaging support surface. In this manner, the laser line emanating from the scanner may sweep along the QA sample and the imaging support surface as the structured light scanner is moved by the robot/actuator.

In one example, the structured light scanner is a SICK® TriSpector 1000 3Dlaser scanner mounted to a linear actuator, such as a belt-driven linear actuator available from Festo Corporation of Islandia, NY. In one specific example, a Festo® toothed belt axis ELGE-TB actuator is used. The linear actuator may include an integrated encoder to track the movement of the scanner for accurately capturing image data relative to sweep distance of the laser line. In addition, the linear actuator as well as the scanner may be I/O link capable for ease of integration.

In other examples, the QA sample may be moved relative to a stationary structured light scanner with a conveyance system, such as with an endless conveyor belt. In such an example, the endless conveyor belt may define the imaging support surface. An encoder may be used to track belt movement for accurately capturing image data relative to sweep distance of the laser line.

In some examples, the image sensor assembly 132 may additionally include a still optical camera, stereo camera, etc., mounted to the linear actuator, such as one of the cameras described above. In such an example, the image sensor assembly 132 can capture still images of a QA sample at various angles in addition to capturing a structured light scan. It can be appreciated that capturing both still images at various angles and a structured light scan can generate even more reliable, complete image data of a QA sample.

In one example, the image sensor assembly 132 may include a robot capable of identifying and retrieving (e.g. picking) a sample of QA samples being conveyed in the processing system 104. For instance, a 3D laser scanner (such as the SICK® TriSpector 1000 3Dlaser scanner) may be secured on an end of a robot arm also having grippers, and the robot may move the 3D laser scanner over a QA sample for scanning, pick the QA sample from the conveyor, and place the QA sample on a scale of the QA station 106 to obtain weight data. The 3D laser scan data could be used to generate a 3D model to determine height and volume of the QA sample, which could be combined with the weight data to obtain density. Such an example could simplify a QA station because only a scale would be needed, and scanning while weighing could be avoided. The robot may be a relatively inexpensive robot capable of identifying and retrieving, e.g., a few samples a minute to be place on a QA station platform. For instance, the robot used may be the VIM-303 robot available from Visual Robotic Systems, Inc. of Eugene, OR. In other examples, the robot may be a Universal Robots UR series robot available from Buchanan Automation of Snohomish, WA.

As noted above, the QA scanning system 108 may include a light assembly 136 configured to sufficiently illuminate a QA sample being captured by a camera of the image sensor assembly 132. If a still camera is used, the light assembly 136 may include a light source configured to project a flash of light onto a QA sample and the support surface on which it rests as an image is captured with the camera. In other examples, a light source may produce a constant low level of light that is projected onto a QA sample/support surface. The light source may be one or more of a strobe light (e.g., camera flash) mounted near the camera lens, a ring light surrounding a camera lens, ambient light, etc. The light assembly 136 may further include a shroud assembly or other devices (e.g., air knives, mist evacuations systems, etc.) for sufficiently controlling the lighting conditions, regardless of the environment.

The QA scanning system 108 may further include an image processor 134, which may be used to receive, process, package, and/or send QA image data captured with the image sensor assembly 132 to the QA computing device 111 or another computing device (such as the monitoring system 112, the model management computing device 113, and/or the processor computing device 130 of the processing system 104). In that regard, the image processor 134 may receive still images, 3D laser scan images, etc., and may perform any pre- or post-processing for sending QA image data to the QA computing device 111 or another computing device. For instance, the image processor 134 may send QA image data to the model management computing device 113 for training one or more machine learning models, as discussed herein. Any image data generated by a camera and/or scanner of the QA station 106, whether pre- or post-processed, may simply be referred to herein as “QA image data.”

Pre- or post-processing may include generating views from image data, formatting image data and/or views generated from image data, adding metadata to image data, packaging/condensing/transposing image data for transmitting to the QA computing device 111 or another computing device, etc. For instance, one or more of the imaging and/or calibrating methods described in U.S. Pat. Nos. 8,839,949, 10,471,619, 10,654,185, 10,721,947, 11,475,977, 10,427,882, 10,869,489, 11,266,156, 11,570,998, incorporated by reference in their entirety, may be used for pre-processing. It should be appreciated that some or all of the pre- or post-processing may instead be carried out by the QA computing device 111 or another computing device (such as the model management computing device 113).

In some examples, the image processor 134 may include one or more formatting modules configured to format image data for optimal transport to and/or processing by the QA computing device 111 or another computing device. For instance, formatting modules of the image processor 134 may perform at least one of transforming the image data, re-sizing the image data, labeling the image data, augmenting the image data, etc. In the specific example of a still image, formatting modules may perform at least one of gray-scaling the image, translating the image, rotating the image, scaling/re-sizing the image, adjusting contrast of the image, changing the contrast of the image data, adapting the image to certain model constraints, etc.

In some examples, the image processor 134 may process the 3D laser scan data for generating views/images from 3D scan data. For instance, the image processor 134 may receive 3D laser scan sensor data from the image sensor assembly 132 and generate a 3D point cloud of the scene. The image processor 134 may also package the 3D point cloud data in a suitable format for processing by a computing device, such as for creating a 3D model of a QA sample and/or a 2D representation of the height or elevation of the scene. In that regard, the image processor 134 may package the 3D point cloud data in a suitable format for transmission and/or further processing, such as CSV, .las, .ply, .png, .pdf, or similar. The 3D model of the QA sample and/or a 2D representation of the height or elevation of the scene may instead be generated by another computing device, such as the QA computing device 111.

The image processor 134 may include access to or otherwise be incorporated into a suitable structured light/3D laser scan software program(s) for scanner configuration and setup. The 3D scan software program(s) may be integrated into the 3D laser scanner or accessible on a remote or cloud-based server. If 3D laser scanner is a SICK® TriSpector 1000 3Dlaser scanner, the integrated SOPAS software may be used to configure the scanner.

3D laser scan configuration may include defining parameters for objects, planes, region of interest, fields of view, blobbing (what are you blobbing, size, min width rectangle, etc.), pixel size (e.g., based on encoder counts), sensors, scan trigger points, data pre-processing, data output transmission, etc. For instance, the imaging support surface on which a QA sample rests during scanning (and optionally during weighing) may be defined as a plane. The plane may be used as a reference from which all height measurements may be determined.

Using a task module in the integrated SOPAS software or a similar software tool, the plane may be defined by using a predetermined percentile of points that are flat when scanned (e.g., the top 6%), ignoring an area in which a QA sample is normally located on the surface (e.g., a circle substantially centered on the surface). A plane can be built from those points. The plane may be ascertained for each 3D scan either with or without a QA sample resting on the imaging support surface. In this manner, any height measurements of a QA sample are always effectively calibrated because they may be calculated with reference to the imaging support surface plane for each scan image.

In other examples, a plane representing the imaging support surface may be calculated as parallel to a background in a still camera depth image using one or more plane equations, such as after a black background of an imaging surface is defined as a region of interest (ROI) for the image. Aspects or features of a background in a depth image may be used to obtain pixel samples of the background, such as by defining four corners or portions of the background. The plane can be calculated as substantially parallel to the four corners or portions of the background (e.g., a mean of pixels may be used to estimate a plane if not flat). After defining the plane, a 3D affine transformation matrix may be calculated to enable the image of a QA sample to be rotated and translated as a 3D object on top of the background plane.

After scanning, the image processor 134 may execute one or more modules (e.g., in SOPAS) to blob out the QA sample and separate it from the plane. In one example, an image segmentation machine learning model may be used to separate the QA sample (e.g., shown as a color image) from the plane (e.g., shown as a black background). For instance, the image segmentation machine learning model may incorporate the Segment Anything Model (SAM) available from Meta AI, FastSAM from Ultralytics, or another suitable image segmentation model using image segmentation techniques.

Each QA project may be identified as a “job” by correlating the scan and still image data to the QA sample plant number, system line/machine number, sub-lane number of the processing system, QA bin number, product sku or serial number, QA machine number, date/time, etc. In that regard, the image processor 134 may add metadata to any QA image data to indicate an origin of the QA sample being scanned/imaged.

The image processor 134 may format or package the QA image data for sending to the QA computing device 111 or another computing device either automatically when acquired by the camera, scanner, etc., in response to a request, and/or upon a trigger, such as when other QA data (e.g., weight data) for a specific QA sample or job is ready for sending. For instance, for each scan and still image, the QA image data may be exported to the QA computing device 111 or another computing device through a command channel. In that regard, the image processor 134 may be configured with an output interface for sending the QA image data to the QA computing device 111 or another computing device, such as ftp. In one example, the image processor 134 may output a .png file containing image data (e.g., FRS (reflectance) data, height data (peak height, min height, etc.), encoder data, etc.) along with any command strings and any other data to the QA computing device 111 or another computing device.

As noted above, the QA station 106 may further include a weight measurement assembly 110 configured to gather QA weight data of a QA sample. In general, the weight measurement assembly 110 is configured to capture QA weight data of a QA sample that may be used to assess one or more attributes of the QA sample for QA analysis, such as its weight, density, and/or volume. The QA computing device 111 may then use the QA weight data to determine, for instance, whether the QA sample is within spec.

Examples of a weight measurement assembly 110 for use in the QA station 106 will now be described. FIG. 1 depicts a block diagram that illustrates aspects of a non-limiting example of the weight measurement assembly 110 according to various aspects of the present disclosure. In the depicted example, the weight measurement assembly 110 includes a weight input device 138 for capturing QA weight data and an output device 140 for outputting the QA weight data to a processing device for QA analysis. For instance, the output device 140 may output the QA weight data to the QA computing device 111 through suitable wired or wireless means, such as through an ethernet, serial, or USB connection, Bluetooth, etc. In some examples, the QA weight data may be manually outputted (or inputted into) to the QA computing device 111 by a technician.

The weight input device 138 may be any suitable weight measurement device suitable for capturing a substantially accurate weight of the QA sample, whether the QA sample is stationary or being moved by a conveyance system. In one example, the weight input device 138 is a simple bench scale, such as an analog or digital scale (e.g., an iScale) that is configured to capture an analog or digital weight reading of a QA sample. In one example, the weight input device 138 is a high precision platform or bench scale having an integrated load cell and controller, optionally of food grade, stainless steel. The load cell may have an accuracy up to a tenth of a gram or the like depending on the application. Preferably, the scale is I/O capable for ease of installation and use.

The platform of the scale may also define the imaging support surface on which a QA sample is placed for being imaged by the image sensor assembly 132. In that regard, the QA sample can be weighed while simultaneously being imaged/scanned by the image sensor assembly 132. To support accurate imaging/scanning, the platform of the scale may preferable remain substantially stationary (not move in the vertical direction) when supporting a QA sample, especially if calibration of the scale platform is done before weighing and imaging the QA sample as described above. In other examples, calibration of the scale platform may be done when the QA sample is on the platform, such as by defining the platform as a plane in the scanner configuration as described above. In such an instance, scale platform movement after receiving a QA sample would not adversely affect simultaneous imaging.

In some examples, the weight input device 138 may be a support device that moves in response to the weight of a QA sample, and the degree of movement of the support device is measured by the image sensor assembly 132. For instance, the imaging support surface may be supported by high precision springs that allow for vertical movement under the increased load/weight of a QA sample. The vertical movement captured by the image sensor assembly 132 may be translated into a weight measurement for the QA sample by the image processor 134 and/or the QA computing device 111.

In some examples, the weight input device 138 may be incorporated into a conveyance system having a conveyor belt that moves product over the weight input device 138. For instance, the weight input device 138 may be configured as a load or weigh cell positioned beneath a belt portion of a conveyor belt such that when a QA sample passes over the weigh cell, a weight measurement for the QA sample may be taken. In that regard, the weight input device 138 may be configured as a weigh deck. For situations where a weigh deck is already in place at a food processing plant for weighing/sorting products processed by a processing machine, data captured by the weigh deck may be sent to the QA computing device 111 or another computing device for QA analysis and processing (such as the model management computing device 113 for training one or more machine learning models and/or the monitoring system 112 for QA weight analysis and reporting).

In some examples, the weight input device 138 may be defined by the conveyor belt itself, wherein vertical displacement of the belt caused by the weight of the QA sample may be measured to determine the product weight. For instance, vertical displacement of the conveyor belt may be captured in images by the image sensor assembly 132 and thereafter processed by the image processor 134 or the QA computing device 111 to determine the product weight.

If the image sensor assembly is placed above a vertically displaceable imaging support surface for measuring weight and the vertical displacement of the surface is not used for measuring weight, the QA station 106 may be configured to accommodate for such vertical movement when processing the QA image data. For instance, in some examples, the image sensor assembly may move vertically with the imaging support surface such that the distance between the image sensor assembly and the imaging support surface remains substantially constant. In other examples, the image processor 134 and/or the QA computing device 111 may account for such vertical displacement of the imaging support surface (such as by running a dynamic calibration module). In the examples described herein, the imaging support surface or platform height may be calibrated each time a QA sample is weighed/imaged/scanned, such as by defining the surface as a plane in the scanner configuration and/or in the image.

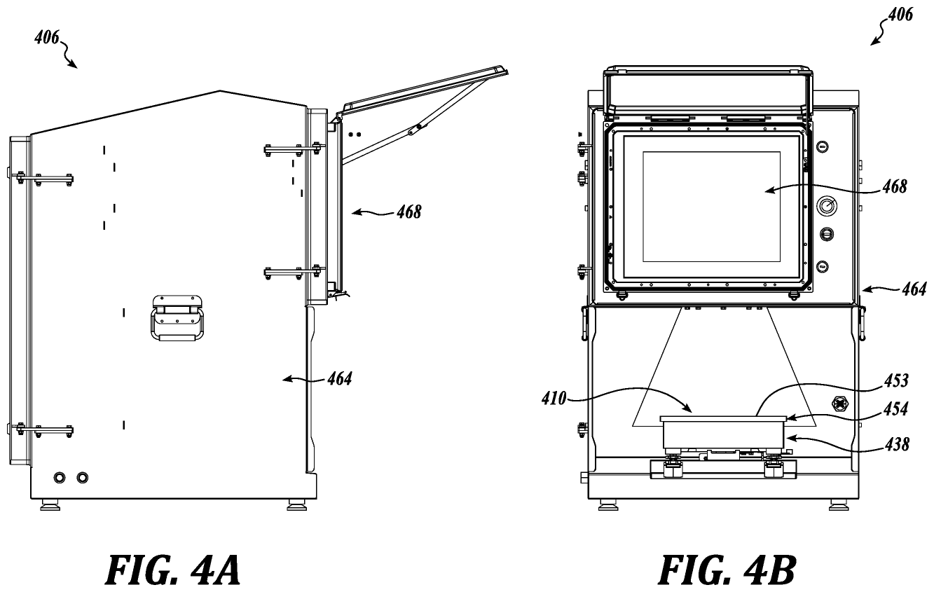

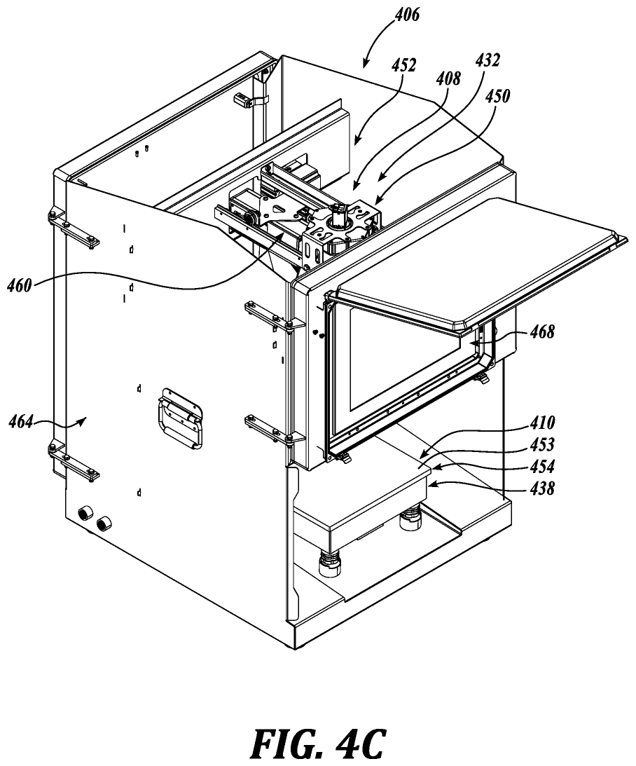

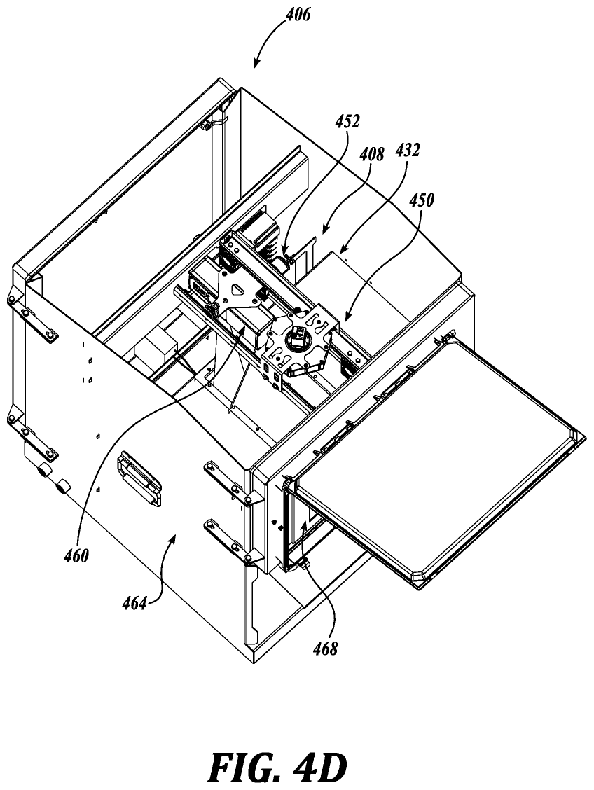

An example of a QA station 406 formed in accordance with systems and methods disclosed herein will now be described with reference to FIGS. 4A-4D. Parts of the QA station 406 like those used in the QA station 106 are labeled with the same reference numeral except in the '400 series. Generally, the QA station 406 includes an integrated QA scanning system 408, weight measurement assembly 410, and at least one QA computing device (not shown).

With an integrated scanning system, weight measurement assembly, and computing device, the QA station 406 may be used to substantially simultaneously and accurately capture image data and weight data. In this manner, fast and accurate QA data may be obtained for performing a QA analysis of a QA sample. For instance, a QA station having an integrated QA scanning system, weight measurement assembly, and computing device, like the QA station 406 shown and described herein, avoids “double handling” of a QA sample, which is prone to error. More specifically, if a QA weight measurement of a QA sample is taken with an assembly or system separate from the QA scanning system, a correlation of data between the weight measurement assembly and the scanning system is needed to confirm that weight and scan data pertains to the same QA sample. Any discrepancy in data sequencing can cause errors, leading to invalid and unusable QA data.

The QA station 406 shown and described herein is one example of a QA station having an integrated QA scanning system, weight measurement assembly, and computing device(s). It should be appreciated that other configurations are possible.

The QA scanning system 408 of the QA station 406 will first be described. The QA scanning system 408 may include an image sensor assembly 432, a light assembly (not shown), and at least one image processor. A housing 464 may enclose at least some of the components of the QA station 406 for sanitation, ease of cleaning, ease of use/storage, etc.

The image sensor assembly 432 may include at least one still camera 450 and at least a structured light scanner 452 located above an imaging support surface 453 on which the QA sample may rest during imaging. The at least one still camera 450 may be mounted to an interior portion of the housing 464 or a structure within the housing. The at least one still camera 450 may be any suitable optical still camera(s) for the intended application, such as one or more of a greyscale camera, an RGB camera an IR camera, a UV camera, a CCD, multispectral or hyperspectral camera, etc.

The light assembly, which may also be mounted to an interior portion of the housing 464 or a structure within the housing, may be any suitable light assembly configured to provide sufficient lighting of the QA sample and imaging support surface 453 for capturing images by the at least one still camera 450. For instance, the light assembly may include a strobe light configured to illuminate the imaging support surface 453 when a snapshot is taken by the at least one still camera 450. In some examples, the at least one still camera 450 may be eliminated, in which case the light assembly may also be eliminated.

The structured light scanner 452 may also be mounted to an interior portion of the housing 464 or a structure within the housing. More particularly, the structured light scanner 452 is mounted to a linear actuator 460 that is mounted to an interior portion of the housing 464 or a structure within the housing. In this manner, the structured light scanner 452 is movable relative to the imaging support surface 453 such that a laser line of the scanner may sweep across a length of the imaging support surface 453. The linear actuator 460 may include an integrated encoder to track the movement of the structured light scanner 452 for accurately capturing image data relative to sweep distance of the laser line. In one example, the structured light scanner 452 is a SICK ® TriSpector1000 3Dlaser scanner and the linear actuator is a belt-driven linear actuator, such as the Festo® toothed belt axis ELGE-TB actuator with encoder.

The structured light scanner 452 may include an integrated image processor configured to run scanner software configurable for the intended application. The scanner software may be used to configure the structured light scanner 452 for the intended application, such as by defining parameters for objects, planes, region of interest, field of view, blobbing, pixel size, sensors, scan trigger points, etc. Configuration may include using a task module in the software to define a plane for the top surface of the imaging support surface 453 such that any height data and measurements may be determined with reference to the plane, as discussed above. Configuration may include using a task module in the software to set up any data pre-processing, data output transmission commands, etc. If a SICK® TriSpector 1000 3Dlaser scanner is used, the integrated SOPAS software may be used to configure the scanner as discussed above.

The weight measurement assembly 410 of the QA station 406 will now be described. The weight measurement assembly 410 may include a weight input device 438 generally configured as a high accuracy bench scale or load cell for capturing QA weight data. In the example shown, the load cell, which defines the platform 454, is positioned on adjustable legs and is removable from an interior compartment of the housing 464. However, in other examples, the weight input device 438 includes a load cell built into a bottom portion of the housing 464. In any event, if the weight input device 438 is intended for use in an industrial food processing setting, it may be washable and made from food grade materials. To help with cleaning, the platform 454 of the scale as well as any interior horizontal portions of the housing 464 may be at a slight angle to facilitate drainage from a front opening of the housing 464.

The weight measurement assembly 410 further includes an output device (not shown) for outputting the QA weight data to a processing device for QA analysis. The output device may be configured as a controller that is separate from or integrated into the weight input device 438. The output device may be configured to perform any pre- or post-processing of weight data and may send it to the QA computing device 111 and/or the processor computing device 130 though suitable wired or wireless means, such as through an ethernet, serial, or USB connection, Bluetooth, etc.

As noted above, the QA station 406 is configured to substantially simultaneously capture image and weight data. In other words, scans and still images of the QA sample may be captured by the image sensor assembly 432 when the QA sample is being weighed or while the QA sample is on the platform 454. In that regard, the platform 454 of the weight input device 438 also defines the imaging support surface 453. The platform 454 of the weight input device 438 may therefore be located beneath the image sensor assembly 432, such as beneath a window defined in an upper portion of the housing 464 through which imaging may occur. In this manner, when a QA sample is placed on the platform 454 to be weighed, the at least one still camera 450 and the structured light scanner 452 may capture still image and 3D laser scan data. Substantially simultaneously capturing image and weight data speeds up a QA process, such as compared to systems that include separate weight and imaging assemblies.

The at least one QA computing device of the QA station 406 will now be described. The at least one QA computing device may be defined by a single device or a collection of devices. For instance, the at least one QA computing device may incorporate one or more functional aspects of the QA computing device 111, described further below, and/or the processor computing device 130. In addition, the at least one QA computing device may include any necessary controllers, adapters, etc., of any of the components of the QA scanning system 408 and the weight measurement assembly 410. Of note, the at least one still camera 450, structured light scanner 452, linear actuator 460, and weight measurement assembly 410 may be I/O link capable to allow for ease of installation and control. Any wired connections between components may be either enclosed inside the housing 464 or passed through a suitable seal received in a housing wall such that the QA station 406 is washdown compatible.

The QA station 406 may include a display unit 468 secured within an upper portion of the housing 464. The display unit 468 may be configured to display information to a user through a graphical user interface (GUI), such as a GUI associated with the scanner software (e.g., SOPAS) or another computing device (e.g., the QA computing device 411 or processor computing device 130). The display unit 468 may be coverable/sealable by a cover to ensure washdown capability.

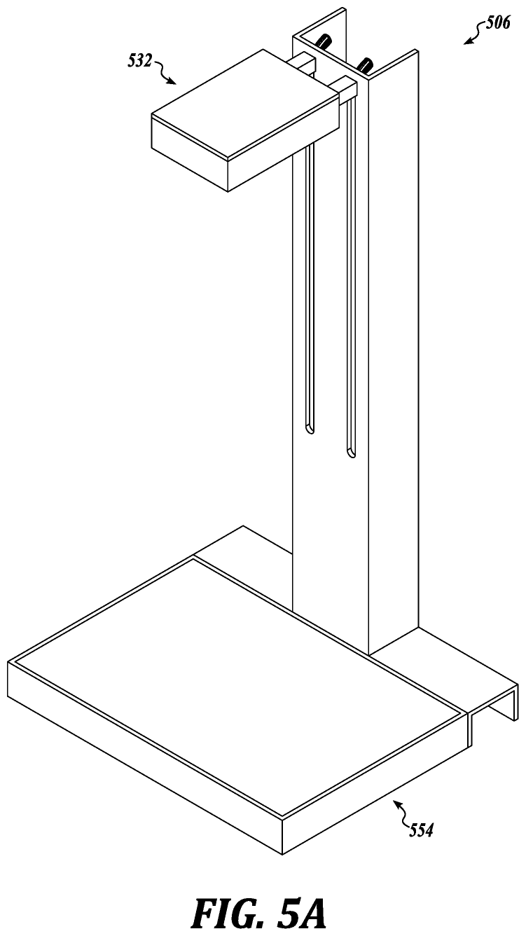

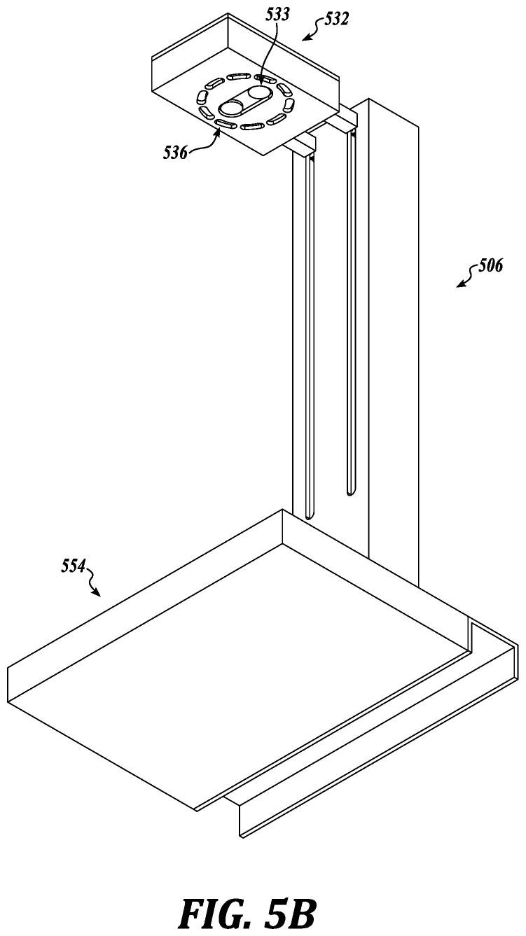

Alternative examples of QA stations 506 and 606 are shown in FIGS. 5A-5B and 6, respectively. Referring to FIGS. 5A and 5B, wherein parts like those used in the QA station 106 are labeled with the same reference numeral except in the '500 series, the QA station 506 may include a platform 554 on which a QA sample to be analyzed for QA may be placed. An image sensor assembly 532 may be positioned above the platform 554 such that its lens(es) 533 are directed downward toward the platform 554 to capture a plan view of the QA sample. The distance between the image sensor assembly 532 and the platform 554 may be adjustable with any suitable height adjustment assembly. A suitable light assembly 536 may be positioned relative to the image sensor assembly 532 for sufficiently illuminating the camera field of view. For instance, a ring light may surround the downwardly aimed lenses of the image sensor assembly 532 to sufficiently illuminate the camera field of view containing the QA sample.

A single image sensor, such as an optical still camera, a stereo camera, etc., may be used in the example shown in FIGS. 5A and 5B for capturing images of a still product. However, it should be appreciated that in some examples, more than one image sensor may be needed. For instance, second and third image sensors may be placed near the first image sensor to capture alternative views of the QA sample, for instance, for creating a more accurate 3D model of the product. If a single camera looking down at the QA sample is used, voids and undercuts beneath the product may not be detected in the image. Additional sensors may be used to view the QA sample at an oblique angle, thereby capturing, for instance, views of the product that may reveal any voids or undercuts beneath the product.

In addition or in the alternative, the single image sensor, such as an optical still camera, a stereo camera, etc., may be moveable relative to the QA sample for capturing multiple views of the QA sample. As discussed above, an image sensor may be mounted to an end of a robot arm, to a linear actuator, etc.

FIG. 6 shows a QA station 606 (where like parts are labeled with like reference numerals except in the '600 series), wherein the QA station 606 includes a moving platform or conveyance system for supporting a QA sample as it passes an image sensor assembly 632 and a weight measurement assembly 610. The image sensor assembly 632 may include a suitable number of image sensors fixed relative to the conveyance system for sufficiently capturing images of the QA sample as it is moved past the image sensor assembly 632. In other examples, the image sensor assembly 632 may include at least one image sensor placed at the end of a robot arm such that numerous still images may be taken of the QA sample and/or its surroundings as the QA sample is conveyed.

A weight input device of the weight measurement assembly 610 may be located beneath the image sensor assembly 632 such that images of the QA sample may be captured during weight measurement, and/or before and/or after the weight measurement is taken. In one example, the weight input device is configured as a load or weigh cell positioned beneath a belt portion of a conveyor belt such that when a QA sample passes over the weigh cell, a weight measurement for the QA sample may be taken. In that regard, the weight input device may be configured as a weigh deck. In some examples, the weight input device 638 may be defined by the conveyor belt itself, wherein vertical displacement of the belt caused by the weight of the QA sample may be measured (such as through images) to determine the product weight.

As noted above, the processing system 104 may be configured to carry out processing of the QA sample before the QA sample is scanned/weighed by the QA station. In that manner, the quality of workpieces processed by the processing system 104 (such as whether the workpieces are in spec) may be analyzed with data gathered from the QA station. For instance, with reference to FIGS. 2 and 6, some or all of the QA samples processed by the processing system 104 may be sorted by the sorter 126 or picked by the pick-up station 124 and directed to QA station 606, such as though a singulator 620. The singulator 620 may be configured to sequentially position a plurality of QA samples for analysis by the QA station 606, such as some or all of the workpieces. In that regard, the QA stations described herein may be incorporated into a food processing line such that some or all of the workpieces processed by the processing system 104 may pass through the QA station for QA analysis after processing.

In some examples, the QA station may additionally or alternatively be located upstream of the processing system 104 for gathering QA data relevant to the incoming workpieces. For instance, in the example of FIG. 6, some or all of the workpieces to be processed by the processing system 104 may be loaded onto the singulator 620 for imaging/weighing by the QA station 606 (and thus are “QA samples”). After passing the QA station 606, the QA samples are manually or automatically loaded onto the conveyance system 116 of the processing system 104 for processing. In other examples, such as with a stationary QA station like QA stations 406 and 506, the QA samples may be manually placed at the QA station and thereafter manually or automatically loaded onto the conveyance system 116 of the processing system 104 for processing.

In some examples, the QA station may be located upstream and/or downstream of a thermal processing system or cooking line having an oven, fryer, steamer, roaster, etc. The QA station may be used to gather QA image data as well as QA weight data for assessing food safety (e.g., has the QA sample been sufficiently cooked), food quality (e.g., does the QA sample color indicate lack of browning, burning, etc.), or other attributes. For instance, the QA station may be located upstream of an oven, and images of an incoming QA sample may be captured by the image sensor assembly for generating a height map or 3D model of the product. In some instances, the incoming QA sample may also be weighed by the weight measurement assembly to acquire weight data. In other instances, an incoming QA sample is weighed only if it is estimated to have a certain minimum volume, as determined from the height map/3D model.

Based on an analysis of the QA image data and/or the QA weight data, a temperature of a QA sample may be taken during and/or after the cooking process. For instance, QA samples that are above a certain volume and/or weight may be assessed during and/or after the cooking process to determine if the QA sample has been properly cooked. By knowing the volume and optionally weight of a thermally treated QA sample, adjustments to the cooking process may be made based on the measured temperature. In addition or in the alternative, adjustments to the cooking process may be made based on the color of the product detected in the color images generated by the image processor.

The method of selecting QA samples to be measured, as well as the temperature measurement and analysis performed on the thermally treated QA samples may be done in accordance with the systems and methods described in U.S. Pat. No. 9,366,579, entitled “Thermal process control”; U.S. Pat. No. 9,366,580, entitled “Thermal measurement and process control”; and U.S. Provisional Patent Application No. 63/517,204, entitled “Programmed Food Equilibration System And Method For Real Time Process Yield In A Thermal Process”, the entire disclosures of which are incorporated by reference herein. The image data from image sensor assembly 132 (and optionally the weight data from weight measurement assembly 110) may be used for selecting the QA samples to be measured (as opposed to or in addition to, for instance, using the processing machine scan data as discussed in U.S. Pat. Nos. 9,366,579 and 9,366,580).

If the QA station is located downstream of a thermal processing line, in addition to an image sensor assembly and optionally a weight measurement assembly, the QA station may be configured to include a temperature probe assembly that is configured to measure and output a temperature of a thermally treated QA sample. For instance, a temperature probe assembly like that described in U.S. Provisional Patent Application No. 63/517,204 may be used. Temperature data may be sent to the QA computing device 111, a computing device of the thermal processing system, and/or another device in communication therewith such that appropriate thermal processing adjustments may be made to ensure proper cooking. In some instances, temperature data may also or instead be sent to the monitoring system 112 for thermal processing QA analysis and reporting.

In some instances, a QA station may be located upstream and/or downstream of a thermal processing line to identify QA samples or pieces that are suitable and/or unsuitable for the thermal process. For instance, pieces that are too large or thick may not sufficiently cook during the thermal process. Similarly, if pieces are stacked together, the stacked pieces may define a combined overall piece size that will not sufficiently cook during the process.

In that regard, the image sensor assembly of a QA station may be positioned relative to a conveyance system moving pieces into and/or out of a thermal processing system, such as an oven, fryer, etc., to identify pieces unsuitable (e.g., too large) for the thermal process. QA image data captured by the image sensor assembly may be used to locate any pieces, for instance, that are larger than a maximum size suitable for thermal processing (e.g., they would not sufficiently cook during the process). In some examples, QA image data generated for some or all of the incoming pieces may be used to generate a height map of the imaged products, and based on a height map analysis (generated, for instance, by the QA computing device 111 and/or the processor computing device 130), pieces exceeding a certain volume are picked from the line and/or diverted from the thermal processing. In some instances, the QA image data generated for some or all of the incoming pieces may exclude any data below a certain height or distance from the image sensor assembly based on, for instance, settings in the image processor. In this manner, only product that is potentially larger than a predetermined volume, based on a minimum height of the product, will be analyzed.

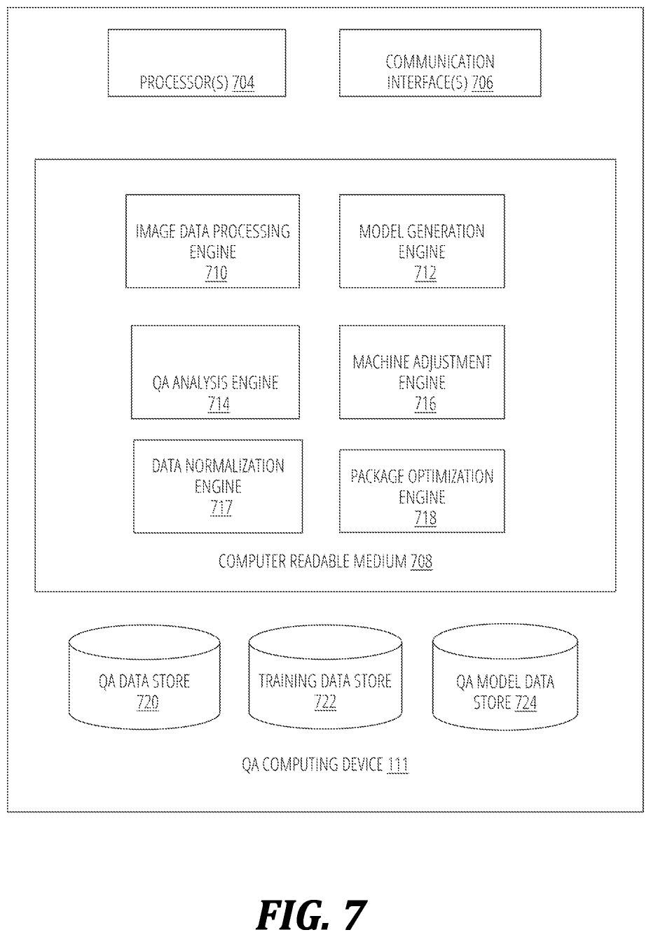

FIG. 7 depicts a block diagram that illustrates aspects of a non-limiting example of a QA computing device 111 configured to carry out some or all of the functions of the QA systems and method described herein. It should be appreciated that certain functions may instead be carried out by another computing device, such as the image processor 134 of the QA scanning system 108, the processor computing device 130 of the processing system 104, or both, and/or another computing device, such as the monitoring system 112 and/or the model management computing device 113. The QA computing device 111 may be in networked communication with any of the other devices of the QA sample processing management system 102, such as the image processor 134 of the QA scanning system 108, the output device 140 of the weight measurement assembly 110, the processor computing device 130, the monitoring system 112, and the model management computing device 113. Although the QA computing device 111 is described with reference to the QA station 106, it should be appreciated that the function of the QA computing device 111 may be incorporated into any suitable QA station, such as QA station 406, QA station 506, and/or QA station 606.

In general, the QA computing device 111 includes a processor(s) 704, a communication interface(s) 706, and computer readable medium 708, and one or more data stores (e.g., a QA data store 720, a training data store 722, and a QA model data store 724).

The QA computing device 111 may be implemented by any computing device or collection of computing devices, including but not limited to a desktop computing device, a laptop computing device, a mobile computing device, an edge computing device, a PLC, a server computing device, a computing device of a cloud computing system, and/or combinations thereof. In some examples, the processor(s) 704 may include any suitable type of general-purpose computer processor. In some examples, the processor(s) 504 may include one or more special-purpose computer processors or AI accelerators optimized for specific computing tasks, including but not limited to graphical processing units (GPUs), vision processing units (VPTs), and tensor processing units (TPUs).

In one example, the QA computing device 111 may be configured as a NVIDIA Jetson Orin package, such as an Advantech MIC-711-OX. A TCP/IP connection may be used to transfer data between the QA computing device 111 and the processor computing device 130 and/or another computing device (e.g., the monitoring system 112 or the model management computing device 113).

In some examples, data speed between the QA computing device 111 and the processor computing device 130 and/or another computing device (e.g., the monitoring system 112 or the model management computing device 113) may be increased by using PCI firewire or other communication bridges. In other examples, data speed between the QA computing device 111 and other computing devices may be increased by continuing to use a general network protocol connection like TCP/IP, but while increasing the processing power of the QA computing device 111.

A communication protocol may be used to reliably and efficiently send data between the QA computing device 111 and other computing devices. The communication protocol may be configured as a high-level protocol that is not platform dependent and allows for simple commands to be used. For instance, the communication protocol may enable two-way communication between the QA computing device 111 other computing devices. Protocol Buffers (Protobufs) may be utilized to optimize efficiency of data transport. The protocol may support both synchronous and asynchronous communications. In some examples, a high level, restricted API implemented on the QA computing device 111 and/or the processor computing device 130 may be used to verify transmitted sensor data.

In some examples, the communication interface(s) 706 include one or more hardware and or software interfaces suitable for providing communication links between components. The communication interface(s) 706 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof.

As shown, the computer readable medium 708 has stored thereon logic that, in response to execution by the one or more processor(s) 704, may cause the QA computing device 111 to provide an image data processing engine 710, a model generation engine 712, a QA analysis engine 714, a machine adjustment engine 716, a data normalization engine 717, and a package optimization engine 718.

Exemplary aspects of the image data processing engine 710 will first be described. The image data processing engine 710 may be generally configured to process, format, store, and/or transmit any image sensor data received and/or retrieved from the image sensor assembly 132 and/or the image processor 134 of the QA scanning system 108. As noted above, the QA scanning system 108 may include an image processor 134, which may receive still image and 3D image sensor data from the image sensor assembly 132. The image processor 134 may package and send the image sensor data (“QA image data”) to the image data processing engine 710, which may process the QA image data, store the QA image data in the QA data store 720 for later retrieval, and/or transmit the QA image data to another device or engine (e.g., the model generation engine 712).

In some examples, the image data processing engine 710 may include circuitry for generating views/images from the scan data and/or processing data from the different views (in addition to or instead of the image processor 134). For instance, the image data processing engine 710 may be configured to generate at least one of a fat recognition (FRS) object view of a QA sample, a laser scatter object view of a QA sample, and a height mode object view of a QA sample from the QA image data (e.g., a 2D representation of the height or elevation of the scene). The image data processing engine 710 may include circuitry for executing one or more feature recognition modules for identifying features of the QA sample, such as object outline, shape, color defects (e.g., for blood streaks), striation, etc. In that regard, metadata may be added to a file to describe any identified features (e.g., tags, descriptions, etc.). Metadata may be used for identifying a workpiece in a data set, training one or more machine learning models used for QA analysis and/or workpiece processing, etc. The image data processing engine 710 may also process the QA image data to generate a height map for producing a 3D model of the QA sample.

The image data processing engine 710 may be configured to eliminate any background image data when generating QA image data. In that regard, the image data processing engine 710 may be programmed to exclude image data of a certain color (e.g., if the surface on which the product is imaged is of a certain color) and/or image data at a certain depth (e.g., the known distance of the surface relative to the camera). Background data or other data may instead be excluded from data processing in any other suitable manner. For instance, in addition or in the alternative, an imaging support surface (see, e.g., imaging support surface 453 shown in FIGS. 4B and 4C) may be calibrated for each scan (e.g., defined as a reference plane) by defining the imaging support surface as a plane using the 3D laser scanner integrated software (e.g., SOPAS) or plane calculation method as described above. In this manner, anything below the plane can be eliminated when processing QA image data (e.g., the plane is defined or otherwise calibrated for each scan).

A QA sample isolation image segmentation machine learning model, such as that described in U.S. Provisional Patent No. 63/588,917, entitled “Edge Computing Device System And Method”, may also be used to isolate a QA sample on an imaging support surface 453. Using a QA sample isolation image segmentation machine learning model may be useful when an imaging support surface is uneven. In that regard, a QA sample isolation image segmentation machine learning model may improve upon known methods of accounting for an uneven bottom surface of a QA sample (e.g., voids), such as those described in U.S. Pat. No. 11,570,998, incorporated by reference herein. The method described in U.S. Pat. No. 11,570,998 can be used to account for an uneven bottom surface of a product in calculating height or weight/mass of a QA sample. However, such methods do not account for an uneven belt surface. Thus, the method described in U.S. Pat. No. 11,570,998 may be improved by using a QA sample image segmentation machine learning model described herein to account for belt height differences.

The image data processing engine 710 may be configured to generate an image of a QA sample having multiple channels or layers. For instance, the image data processing engine 710 may combine image files into a file(s) with one or more corresponding channels or layers. For instance, an FRS image and a height map image may be combined into a single file with two channels. By combining images into a single file having multiple channels, all the necessary image data can be sent to the model generation engine 712 or another computing device in a single file rather than in separate files. As such, the QA computing device 111 or another computing device can process the file at optimal speeds and with higher accuracy.

In some examples, the image data processing engine 710 may include one or more formatting modules configured to format QA image data for optimal transport to and/or processing by another engine of the QA computing device 111 or another computing device, such as one or more of the processor computing device 130, the monitoring system 112, and the model management computing device 113. For instance, formatting modules of the image data processing engine 710 may perform at least one of transforming QA image data, re-sizing the QA image data, labeling the QA image data, augmenting the QA image data, etc. In the specific example of an image, formatting modules may perform at least one of gray-scaling the image, translating the image, rotating the image, scaling/re-sizing the image, adjusting contrast of the image, changing the contrast of the image data, adapting the image to certain model constraints, etc. Any suitable image processing libraries (e.g., Python) available to the QA computing device 111 may be used to process or format the image data. Processed/formatted QA image data may be saved to the QA data store 720, sent to another engine of the QA computing device 111, and/or sent to another computing device, such as one or more of the processor computing device 130, the monitoring system 112, and the model management computing device 113.

In one example, the image data processing engine 710 sends processed/formatted QA image data for a QA sample to the model management computing device 113 or a computing device (e.g., a cloud-based computing device) in communication with the model management computing device 113. The QA image data may be used to train one or more machine learning models executable by the QA computing device 111 for QA analysis and/or the processor computing device 130 for workpiece processing and/or machine management. In that regard, the same or substantially similar processing/formatting may be done for any data used for both training and using the machine learning models for optimal consistency, reliability, and speed.

In one example, the image data processing engine 710 (and/or the image processor 134) may use one or more image processing modules and/or machine learning models trained to identify the QA sample for QA analysis. For instance, if a variety of portioned or cut pieces are being conveyed on a belt, the image data processing engine 710 may be configured to identify the pieces required for QA analysis.

In some examples, the image data processing engine 710 may run an image data optimization module to select conflicting/competing sensor data (e.g., 3D point cloud data from) originating from different image sensors and/or sensor sources. As discussed above, the image sensor assembly 132 may include more than one type of sensor assembly and/or a plurality of sensors of the same type, such as for capturing images showing various views of the product to help generate an accurate 3D or 2D model. For instance, the image sensor assembly 132 may include at least one of a 3D vision system or a 3D laser scanning technology like LiDAR (Light Detection and Ranging), structured light scanning, or photogrammetry, a stereo camera, a Time-of-Flight (ToF) stereoscopic camera, or the like, or combinations thereof. In that regard, if more than one sensor or more than one type of sensor is used, conflicting/competing sensor data (e.g., sensor data pertaining to the same 3D point cloud data of the product) may be captured for the QA sample.

In that regard, the image data processing engine 710 may run an image data optimization module to select conflicting/competing sensor data. The image data optimization module may select 3D point cloud data, for instance, depending on the resolution of the image sensor data, the processing power required to use the image sensor data, the compatibility of the image sensor data with other modules, etc.

The 3D point cloud data selected may also depend on the intended use of the 3D or 2D model. For instance, if a 3D model is being generated for determining the height and/or volume of a QA sample, such as for combining with a weight measurement to determine density of the product, the 3D point cloud data selected may include 3D laser scan data. If a 3D model is being generated for determining the surface texture of a QA sample, the 3D point cloud data selected may include stereo camera data (which may produce clearer images of texture). In that regard, different types of sensor data may be selected to generate one or more 3D or 2D models showing different attributes of the QA sample.

In some examples, the image data optimization module of the image data processing engine 710 uses one or more machine learning models (e.g., stored in the QA model data store 724) to identify 3D point cloud data from conflicting/competing sensor data as output based on the accuracy/efficiency/etc., of the model generated for use in a QA analysis as input. For instance, when a sensor data type is selected for generation of a certain type of model (e.g., a 3D model showing volume), the sensor data used for that model can be stored in the QA data store 720 and categorized based on the accuracy/efficiency/etc., of the model for use in a QA analysis (such confirmed by secondary manual testing or other criteria). The machine learning models may be trained to select 3D point cloud data for each model using model accuracy/efficiency/etc., data or other criteria as training data. The selected 3D point cloud data and/or the 2D or 3D models generated with the 3D cloud point data may be stored in the training data store 722 and/or sent to or retrieved by the model generation engine 712 for training the machine learning models.

It should be noted that any aspects of the image data processing engine 710 may instead or additionally be carried out by the model generation engine 712.

Exemplary aspects of the model generation engine 712 will now be described. The model generation engine 712 may be generally configured to produce a 2D or 3D model of the QA sample using image data and/or 3D laser scan data (e.g., 3D point cloud data) from the image processor 134, the image data processing engine 710, and/or the QA data store 720. In that regard, the model generation engine 712 may include software modules suitable for processing the image data and 3D point cloud data and generating 3D models (showing contour, shape, volume, texture, etc.), 2D models (e.g., showing a height and outline), or other images. For instance, the model generation engine 712 may run the proprietary DSI Q-LINK™ Portioning Software developed by Design Systems, Inc. of Redmond, Washington.

In the examples described herein, QA weight data for the QA sample may be ascertained using the weight measurement assembly 110 (and in preferred embodiments, the weight is obtained substantially simultaneously with the QA image data as discussed above). In that regard, the model generation engine 712 and/or the image data processing engine 710 may be configured to associate QA weight data with QA image data of the QA sample. QA weight data may be specified in metadata of the image data and/or in any 2D or 3D models generated from the QA image data. In this manner, the weight of the QA sample can be considered together with data in the 2D/3D models when performing a QA analysis of the QA sample. Any other data may also be considered. As noted above, QA data may include image data, weight data, and any other data pertaining to the QA sample that is gathered by the QA system for assessing one or more attributes of the QA sample. For instance, the QA data may include measurements of QA samples and/or conveyor belt components generated by a high-speed optical micrometer, as discussed in U.S. Provisional Patent No. 63/588,917, incorporated herein.

In some examples, the weight measurement assembly 110 may be configured to capture QA weight data for a plurality of related QA samples, such as portioned pieces of a workpiece. It may be beneficial to obtain weight data for each portioned piece of workpiece to ensure the processing system 104 is appropriately portioning each workpiece. For instance, if the processing system 104 is portioning a chicken breast fillet to create chicken nuggets, a quality analysis may include determining whether each of the nuggets have substantially the same shape, weight, and/or size and/or whether they conform to spec. As another example, if a chicken butterfly is portioned into chicken breast fillets, a quality analysis may include determining whether each of the chicken breast fillets have substantially the same weight and/or size.

In one example, the weight measurement assembly 110 may be configured to capture QA weight data for each of the plurality of related QA samples as each of the samples are added to a stationary or displaceable platform of the weight measurement assembly 110. For instance, the weight measurement assembly 110 may generate weight data for each of the plurality of related QA samples as each of the plurality of related QA samples are placed on a weight platform, such as a platform of a bench scale, on a conveyor system having a load cell, on a vertically displaceable surface imaged by the QA station 106, etc. A controller of the weight measurement assembly 110 and/or the QA computing device 111 or another computing device may determine, in real time, an individual weight of each of the plurality of related QA samples using a first total weight having N QA samples and a second, subsequent total weight having N+1 QA samples. For instance, the first total weight may be subtracted from the second, subsequent total weight to determine the weight of the Nth+1 QA sample. In that regard, each of the weights may be stored in a data store of the controller/computing device for use in the weight calculation.

In addition or in the alternative, the weight measurement assembly 110 may be configured to may generate QA weight data for each of the plurality of related QA samples after the controller/computing device of the weight measurement assembly receives an input indicating that a sample will be added to the weight platform. For instance, after an operator hits an “Add” button or similar, a QA sample of the plurality of related QA samples may be added to the weight platform. A weight is captured each time after the “Add” input (or similar), and controller of the weight measurement assembly 110 and/or the QA computing device 111 or another computing device may determine, in real time, an individual weight of each of the plurality of related QA samples. Similar to above, the individual weight of each of the plurality of related QA samples may be determined using a first total weight having N QA samples and a second, subsequent total weight (prompted by the “Add” input or similar) having N+1 QA samples and subtracting the first total weight from the second, subsequent total weight to determine the weight of the Nth+1 QA sample. In that regard, each of the weights may again be stored in a data store of the controller/computing device for use in the weight calculation. In other examples, the weight may be zeroed out each time the “Add” input is received, and the weight for the added QA sample may be obtained and recorded.

In another aspect, either of the above techniques for obtaining QA weight data for each of the plurality of related QA samples may be done by performing the technique in reverse. For instance, instead of obtaining a weight measurement each time a QA sample is added to a weight platform, the weight measurement may instead be obtained by first adding all the related QA samples to the platform, and then obtaining individual measurements as each piece is removed from the platform.

In another example, the weight measurement assembly 110 may be configured to capture a total weight for all of the plurality of related QA samples, and the (optionally simultaneously captured) QA image data may be used together with the total weight to determine QA weight data for each of the plurality of related QA samples. For instance, all of the plurality of related QA samples may be placed on a weight platform (e.g., a platform of a bench scale, on a conveyor system having a load cell, on a vertically displaceable surface imaged by the QA station 106, etc.) and a total weight measurement for all of the samples may be obtained. A total volume for all of the plurality of related QA samples and a piece-by-piece volume for each of the plurality of related QA samples may be obtained using QA image data, such as in accordance with one of the techniques described herein. Knowing the total volume of all the samples and the piece-by-piece volume, the ratio of volume for each of the plurality of related QA samples relative to the total volume of all of the samples may be obtained. That volume ratio may then be used to determine a weight for each of the plurality of related QA samples using the total weight for all of the plurality of related QA samples. In other words, each of plurality of related QA samples will have an individual weight to total weight ratio that is substantially the same as the individual volume to total volume ratio.

In any event, the QA weight data for all of the plurality of related QA samples as well as piece-by-piece QA weight data for each of the plurality of related QA samples may be correlated to QA image data capturing all of the plurality of related QA samples. For instance, QA weight data may be specified in metadata of the image data and/or in any 2D or 3D models generated from the QA image data for all of the plurality of related QA samples. In this manner, the QA weight data for all of the plurality of related QA samples as well as piece-by-piece QA weight data for each of the plurality of related QA samples can be considered together with data in the 2D/3D models of all of the plurality of related QA samples when performing a QA analysis of the QA sample.

To help illustrate this point, if a QA analysis is to be performed for chicken nuggets portioned from a chicken breast fillet, QA weight data for each of the chicken nuggets produced from a chicken breast fillet, as well as the total weight of the chicken breast fillet portioned into nuggets, may be stored and/or correlated to QA image data of the chicken breast fillet portioned into nuggets. Such correlated data may be used to perform a QA analysis for each of the portioned nuggets, such as determining whether each of the nuggets are the correct size, shape, and weight. Such correlated data may also be used to perform a QA analysis of the incoming chicken breast fillets to be portioned into nuggets. For instance, the correlated data may be used to appreciate discrepancies between the expected and measured values of the incoming chicken breast fillets to be portioned into nuggets. Further aspects will become better appreciated in the descriptions below.

In some examples, the model generation engine 712 and/or the image data processing engine 710 may include one or more formatting modules configured to format QA data for optimal transport to and/or processing by the QA computing device 111 or another computing device. For instance, formatting modules of the model generation engine 712 may perform at least one of transforming QA data, re-sizing the QA data, labeling the QA data, augmenting the QA data, etc. Any suitable image processing libraries (e.g., Python) available to the QA computing device 111 may be used to process or format the QA data. Processed/formatted QA data may be saved to the QA data store 720, sent to another engine of the QA computing device 111, and/or sent to another computing device, such as one or more of the processor computing device 130, the monitoring system 112, and the model management computing device 113.

In one example, the model generation engine 712 sends processed/formatted QA data for a QA sample to the model management computing device 113 or a computing device (e.g., a cloud-based computing device) in communication with the model management computing device 113, optionally after being stored in the training data store 722. The QA data may be used to train one or more machine learning models executable by the QA computing device 111 and/or the processor computing device 130. In that regard, the same or substantially similar processing/formatting may be done for any data used for both training and using the machine learning models for optimal consistency, reliability, and speed. In some examples, the one or more machine learning models may be executable by the QA analysis engine 714 of the QA computing device 111.

Exemplary aspects of the QA analysis engine 714 will now be described. The QA analysis engine 714 of the QA computing device 111 may be generally configured to analyze QA data, including 2D and/or 3D models or other image data generated by the model generation engine 712 and any associated weight data for performing a QA analysis of QA samples.

In examples where a QA analysis is being performed for a QA sample for carrying out one or more subsequent actions for that QA sample, the QA analysis engine 714 may first perform a model matching process to confirm that the model or other image data generated by the model generation engine 712 matches the QA sample being analyzed or processed. Such a model matching process may be used, for instance, when incoming product pass through the QA station 106 before being scanned and processed by processing system 104 such that automatic adjustments may be made to the machine as the products are processed (e.g., adjusting the density settings for accurate portioning). Such a model matching process may also be used, for instance, when processed product passes through the QA station 106 after being processed by processing system 104 such that QA image data may be analyzed together with any scan data generated by the processing system 104.

In that regard, the model or other image data generated by the model generation engine 712 may be matched to the scan data of the scanning station 120 of the processing system 104. Matching may be necessary to confirm that the QA sample scanned at scanning station 120 is the same as the QA sample scanned at QA station 106 and/or whether the QA sample has moved or shifted during transfer between conveyors, as discussed in U.S. Pat. Nos. 10,654,185 and 10,721,947 (referenced above), incorporated by reference herein. In that regard, a comparison of the image data may be processed by the QA analysis engine 714.

Matching itself can be performed, for example, by determining discrete locations along the outer perimeter of the QA sample in terms of an X-Y coordinate system or other coordinate system. The QA analysis engine 714 can compare the data identifying coordinates along the outer perimeter of the workpiece as determined by scanning station 120 with corresponding data obtained in the image data generated by model generation engine 712. Matching of image data may include overlaying images to determine a match, and if necessary, the image data may be transformed, e.g., by X-Y translation, rotation, X-Y shear, X-Y displacement or the like. If the data sets match within a fixed threshold level, then confirmation may be provided that the QA sample scanned at QA station 106 is the same as the workpiece scanned at scanning station 120. If no adequate match can be achieved, the workpiece can be skipped in the processing and be transported, for example, to manual processing.

After optionally matching image data, the QA analysis engine 714 may analyze QA data, including models generated by the model generation engine 712 (and/or the image data processing engine 710) as well as any weight data and/or or other image data for performing a QA analysis of QA samples. A QA analysis may include comparing physical parameters/characteristics of a QA sample to a specification for that QA sample. A QA sample specification may include required values for the parameters/characteristics (e.g., minimum, maximum, average, maximum standard deviation, etc.). The QA analysis may be performed to compare any of the physical parameters/characteristics of a QA sample described herein or any other relevant physical parameters/characteristics of a QA sample to a specification for that QA sample.

The specification for a QA sample may be represented as reference data in tabular format, as a 3D model, as a drawing, or as any other suitable format. The specification may be stored for instance, in the QA data store 720 and/or in a data store of the processor computing device 130 or another computing device. The QA analysis engine 714 may retrieve relevant processing specification information stored in the QA data store 720 or another data store for running various modules configured to analyze various aspects of the QA samples.

Based on the results of the QA analysis engine 714, the machine adjustment engine 716 may be used to make or suggest adjustments to the processing machine(s), it may be used to divert the QA sample or any processed workpieces for further processing, it may be used to provide information to a technician or management personnel (such as through a user interface provided by the communication interface(s) 706, such as via a tablet or monitor positioned near the QA station 106), etc. For instance, notifications and/or alarms may be generated if an out-of-spec QA sample is detected.

In one aspect, the QA analysis engine 714 may run a specification module that may include comparing QA image data to specification information for the QA sample. Running the specification module may include identifying coordinates along an outer perimeter of a 2D or 3D model and comparing those coordinates to specifications of the QA sample for assessing the shape, size, specific length/widths, etc., of the QA sample. If the QA image data sets match within a fixed threshold level, then the specification module may indicate that the QA sample conforms to the product specification. If the QA image data sets do not match within a fixed threshold level, then the specification module may indicate that the QA sample is out of spec, and in some instances, how far out of spec (e.g., the percentage of shape non-conformance, the percentage of length difference, etc.).

In some instances, the specification module may include using QA weight data (e.g., captured by the weight measurement assembly 110) to determine whether the weight of the QA sample falls within a required minimum or maximum threshold weight range. For instance, a portioned or trimmed product may be analyzed after processing by the processing system 104 to determine if the processed product is within weight specifications. If the weight of the QA sample is not within a required weight range, then the specification module may indicate that the QA sample is out of spec, and in some instances, how far out of spec (e.g., the percentage of weight difference).

If the QA analysis engine 714 indicates that the QA sample is out of spec, the machine adjustment engine 716 may send instructions to the processor computing device 130 to automatically adjust settings of the processing system 104. For instance, if the QA sample has an incorrect shape, size, or weight, the processor computing device 130 may be instructed to adjust its cutting paths as needed to bring the shape, size, or weight of remaining workpieces to be processed back into spec. In that regard, the adjustments may be made to the machine after analyzing a sampling of products already processed (e.g., with the QA station 106 located downstream of the processing system 104) or after analyzing some or all of incoming product before being processed (e.g., with the QA station 106 located upstream of the processing system 104).

In some instances, the specification module may include using weight data (e.g., captured by the weight measurement assembly 110) to determine a density of the product, and comparing the density value to, for instance, the density value calculated by the processor computing device 130 using scan data of the scanning station 120. In that regard, machine density adjustments may be automatically made, for instance, by the machine adjustment engine 716. Machine density settings may also be adjusted based on the average density of QA samples analyzed by the QA analysis engine 714. In some instances, manual adjustments to the machine may be made based on the average density of QA samples provided by the QA analysis engine 714 (such as through the communication interface(s) 706). In some instances, a notification may be generated indicating a discrepancy in machine density settings v. calculated QA sample densities so that appropriate action may be taken (e.g., determining whether the workpiece has voids or undercutting, large amounts of fat or bone, etc., troubleshooting machine sensors or other components, etc.)

In some instances, the data generated by the specification module or other modules of the QA analysis engine 714 may be used to train one or more machine learning models (e.g., stored in the QA model data store 724) to provide adjustments of the parameters ascertained by processed scan data of the scanning station 120 as output based on data analysis results of the QA analysis engine 714 as input. For instance, when QA analysis indicates that a QA sample has a different density than what was determined by analyzing the scan data of the scanning station 120, at least one of the parameters/settings of the scanning station 120, processing instructions generated by the processor computing device 130 based on results of the scanning station 120, or other machine settings may be automatically adjusted to account for any differences. In some examples, a notification may be provided to operators when QA analysis indicates that a QA sample has a different density than what was determined by analyzing the scan data of the scanning station 120.

In that regard, the machine learning models may be trained, for instance, using training data pertaining to any parameter calculation adjustments, machine adjustments, notifications, etc., that are made or generated in response to a comparison of the QA analysis engine 714 data and the scanning station 120 data (stored in the training data store 722). Such training data may be used to train the one or more machine learning models to output parameter calculation adjustments, machine adjustments, notifications, etc., based on QA data as input.

The QA analysis engine 714 may run various other modules for performing a QA analysis of QA samples. For instance, in some examples, the QA analysis engine 714 may run a product output module that may be used to estimate, for instance, the total volume and/or weight of a production run (the production run “output”), the total volume and/or weight of certain pieces of a production run (e.g., portioned pieces of a certain size), etc. For example, a sensor may be used to count pieces or QA samples passing the sensor on the conveyor belt to generate a piece count rate, and the piece count rate may be multiplied by the average weight of each piece (as determined by QA data generated for at least some of the pieces) to provide a production rate in pounds. As another example, an average area of each piece (as determined by QA data generated for at least some of the pieces) may be combined with belt speed and/or piece count to provide belt coverage data. Data generated by the product output module may be used to determine any relevant production output data.

In some examples, the QA analysis engine 714 may run a texture analysis module that may include comparing texture data taken from the models or other image data generated by the model generation engine 712 to texture specification information for the QA sample (e.g., stored in the QA data store 720 or another data store of the processing system 104). For instance, the texture analysis module may include comparing texture data taken from color images generated by the model generation engine 712 to color scan images taken by the scanning station 120 and processed by the processor computing device 130. For instance, the texture analysis module may run a feature recognition subroutine that is configured to match and/or compare features in each of the images.

Based on a comparison, the texture analysis module may generate an output indicating whether the QA sample is within spec (e.g., sufficient brown color, sufficient char marks, minimum number of blood spots/bruises/fat percentage or streaks/etc.), whether the QA sample has a tendency towards a certain consistency (e.g., striping or a greyish color may indicate woody chicken consistency), or other aspects regarding texture or appearance. In the latter case, the texture analysis module may retrieve data relevant to the volume, shape, size, and density of the QA sample (from the QA data store 720 and/or a data store of the processor computing device 130 containing scan data) to help determine whether the QA sample has a certain consistency. In the specific example of woody chicken, image data pertaining to shape, contour, color, striping, etc., may indicate woody chicken consistency, which may be verified if it has a density value above a certain density threshold. In other instances, a separate probe may be included in the QA station 106 for assisting in the measuring of product consistency (such as its resilience), either before or after it is flagged by the QA analysis engine 714.

The data generated by the texture analysis module may be used by the machine adjustment engine 716 to send instructions to the processor computing device 130 to automatically adjust settings of the processing system 104 if needed, provide a notification to an operator, etc. For example, if the QA sample has a color that indicates that the product is out of thermal processing spec (e.g., insufficient browning or charring), the processor computing device 130 may be instructed to adjust its thermal processor settings to ensure adequate thermal processing. In such an instance, the adjustments may be made to the machine after analyzing a sampling of products already processed (e.g., with the QA station 106 located downstream of the processing system 104).

In another example, if the QA sample has a color or appearance that is out of spec coupled with an abnormal density or resilience value (e.g., indicating possible woody chicken consistency), the processor computing device 130 may be instructed to divert the QA sample, the processed workpieces, and/or the incoming workpieces for a different use and/or additional processing (such as massaging). In such an instance, adjustments may be made to the machine after analyzing a sampling of products already processed (e.g., with the QA station 106 located downstream of the processing system 104) and/or after analyzing some or all of incoming product before being processed (e.g., with the QA station 106 located upstream of the processing system 104).

In some instances, the data generated by the texture analysis module of the QA analysis engine 714 may be used to train one or more machine learning models (e.g., stored in the QA model data store 724) to provide adjustments in machine settings, instructions for machine adjustment, notifications, etc., as output based on texture image data as input. For instance, when QA analysis indicates that a QA sample has a color or texture indicating that is out of thermal processing spec, the models may be used to automatically adjust processing instructions to ensure adequate thermal processing. In that regard, the machine learning models may be trained, for instance, using training data generated from a correlation of the processing instruction adjustments made in response to different QA texture results (stored in the training data store 722). In another instance, machine learning models may be trained, for instance, using training data generated from a correlation of a confirmed QA sample consistency type (e.g., woody chicken) with QA data, such as color image data, density data, and/or resilience data. The machine learning models may be used to estimate a product consistency and automatically adjust machine settings, provide instructions for machine adjustment, send notifications, etc., as output based on QA data as input. For instance, a machine may be instructed to divert the QA sample, processed workpieces, and/or the incoming workpieces for a different use and/or additional processing (such as massaging).

In some examples, the QA analysis engine 714 may train and/or run various other machine learning modules suitable for performing a QA analysis of QA samples, such as one or more of the machine learning modules described in U.S. Provisional Patent No. 63/588,917, incorporated herein. In that regard, the QA computing device 111 may be configured to store and execute machine learning models necessary for processing the QA data.

The machine learning models, as is typical, may require significant processing power and capacity. Moreover, as processing needs change or as machine learning models are improved, it can be appreciated that the ability to easily access, update, and/or upgrade a separate computing device for use with the processing system 104 and optionally one or more additional processing systems in a facility would be beneficial. In that regard, it may be beneficial to configure aspects of the systems and methods described herein as including a QA computing device 111 that is a local, high power or edge computing device separate from the processor computing device 130, such as the data processing computing device described in U.S. Provisional Patent No. 63/588,917. In some examples, the QA computing device 111 is an integrated component of the QA station 106 in wired communication with the components of the QA station 106 (e.g., the image sensor assembly 132, the image processor 134, and the weight measurement assembly 110), such as through an I/O link master.

In some examples, the QA computing device 111 may execute one or more machine learning models that output QA analysis information to the machine adjustment engine 716 and/or the processor computing device 130 using QA data as input. The information may be used to confirm or adjust processing of workpieces of the same or similar type to the QA sample. For instance, the QA analysis engine 714 of the QA computing device 111 may output information regarding a location of a QA sample feature (e.g., bones, sciatic nerve, cut lines, outline, fat or lean area), an outline of the QA sample and any features therein (e.g., bones, fat/lean, foreign objects, etc.), a region of interest of the QA sample (e.g., an area comprising a maximum nominal height of the QA sample), a classification of the QA sample (e.g., sirloin pork chop, center loin pork chop, etc.), a location of a machine component (e.g., a conveyor belt component) relative to a coordinate system, etc.

In some examples, QA computing device 111 may execute one or more machine learning models that output information to the processor computing device 130 or another computing device including information realized by the machine learning model based on measurement values in the QA data. For instance, the QA computing device 111 may output information regarding conveyor belt sag, stretch, wear, etc., based on conveyor belt measurement values in the QA data.

Exemplary machine learning models configured to be carried out by the QA analysis engine 714 will now be described. Some exemplary machine learning model(s) may be substantially similar to those described in U.S. Provisional Patent No. 63/588,917, incorporated herein. Thus, such exemplary machine learning model(s) will only be described briefly for brevity. Moreover, it should be appreciated that the machine learning models described herein are exemplary only, and other variations of the models described and/or additional models may also be used.

In one example, a classification machine learning model may be configured to classify a QA sample as a type of workpiece, such as a type of sub-primal cut to confirm whether the workpieces are being appropriately processed, sorted, packaged, etc. As described in U.S. patent application Ser. No. 18/462,776, hereby incorporated by reference in its entirety, processing sub-primal cuts may vary depending on the type of sub-primal cut. For instance, certain types of sub-primal cuts may be portioned or trimmed in accordance with customer specifications or other requirements specific to that cut type. Moreover, certain types of sub-primal cuts may be used in certain end products depending on, for instance, supply and demand of the types of sub-primal cuts.

Classification machine learning models may be configured to identify QA samples as a type of sub-primal cut and categorize the sub-primal cut type into one of at least two categories, such as for confirming or adjusting value sorting and/or value optimizing of the processed or incoming sub-primal cuts. For instance, a classification machine learning model(s) may be configured to identify sub-primal cuts or “chops” of a full bone-in pork loin, such as those shown and described in U.S. patent application Ser. No. 18/462,776, incorporated herein.

A classification machine learning model(s) for identification/categorization of sub-primal cuts (e.g., “chops” of a full bone-in pork loin) into one of at least two categories may be configured to provide at least one classification probability score for a sub-primal cut based on a QA image data of a sub-primal cut. Based on information in the QA image data, the classification machine learning model(s) may output a classification probability score (percent likely) for one of a number of different pork chop types.

A classification machine learning model may be trained with QA image data of QA samples, wherein each image may be labeled with one or more classification types. Such annotated QA image data of QA samples and other image data of the QA sample of interest (such as data acquired with the use of the systems and methods disclosed in U.S. Provisional Patent No. 63/588,917, incorporated herein) may be sent to the model management computing device 113 for training the classification machine learning model. The classification machine learning model may learn to provide classification probability scores for a QA sample based on features recognized in the QA images compared to the training data. Further details of a classification machine learning model are provided in U.S. Provisional Patent No. 63/588,917, incorporated herein.

In other examples, a QA sample 3D generation machine learning model may be configured to generate a 3D model of a QA sample as output after receiving top and bottom image data of the QA sample from the image sensor assembly 132 as input. For instance, the image processor 134 and/or the image data processing engine 710 may generate image data (e.g., greyscale, height, etc.) of both top and bottom surfaces of a QA sample (e.g., a pork chop) by utilizing a prior cut piece top image of a QA sample as a mirror image of the target QA sample bottom image (e.g., sliced chops of a pork loin). In other instances, the image sensor assembly 132 may include a scanner beneath the imaging support surface for capturing image data of a QA sample bottom. In yet other instances, the QA sample may be flipped over such that top and bottom images of the QA sample may be captured. As non-limiting examples, an operator could manually flip a QA sample at a stationary station (e.g., such as with QA station 406 or QA station 506), a QA sample may be flipped as it is transferred between conveyors located beneath first and second scanners for scanning the first and second respective sides, etc. In any event, an image matching process, such as that described above, may be carried out to match or correlate the images to a QA sample.

In instances where a top and bottom image of a QA sample cannot be obtained, such as for a first chop sliced from a pork loin, the 3D generation machine learning model can predict a 3D model based on training data gathered for first pieces when training the QA sample 3D generation machine learning model, such as with QA image data and/or data acquired with the use of the systems and methods disclosed in U.S. Provisional Patent No. 63/588,917, incorporated herein.

In some examples, the QA sample 3D generation machine learning model may generate a 3D model of a QA sample as output by extrapolating identified features from images of opposite (e.g., top and bottom) surfaces of the QA sample through the body of the QA sample. For instance, the 3D model output can be created to account for interior features, such as based on straight line assumptions between features on opposite surfaces. In other examples, the 3D model output may be generated by extrapolating density data from a top surface down to a bottom surface to estimate the shape of the bottom surface including any voids, such as using techniques described in U.S. Pat. No. 11,570,998, hereby incorporated herein.

The 3D model output of the QA sample 3D generation machine learning model may be used by the QA analysis engine 714 for managing various aspects of QA analysis. In one example, the 3D model output may be used to provide a classification probability score for each face (e.g., top and bottom) of the QA sample. In that regard, the overall or final assigned classification of the QA sample and/or the workpieces may be adjusted based on the higher probability score of the two faces and/or supply or demand information from the package optimization engine 718 for workpieces. The final assigned classification of the QA sample may be used by the QA analysis engine 714 to determine whether the processed or incoming workpieces are being appropriately classified by the processor computing device 130. In related examples, the top or bottom of processed workpieces may be selected for display in packaging based on a confirmed classification of that side of a QA sample (e.g., a higher value classification, such as per the package optimization engine 718, may be chosen for display in the packaging).

In another example, the 3D model output of a QA sample may be used to confirm or adjust cut paths of a workpiece. For instance, cut paths for a workpiece may be based on comparing the same top and bottom face attributes to align fat and lean lines of a QA sample from top to bottom. The 3D model output data of the QA sample can be used in a 2D cutting module of the processor computing device 130 for making any needed adjustments to the cutting of a workpiece (e.g., with the cutter station 122) according to certain specifications (e.g., fat removed, bones excised, no lean trim, combined fat areas, etc.). The 3D model output data of the QA sample can also be used to adjust portion cutting and/or trimming of workpieces into a desired overall shape. Adjustments may be made by the machine adjustment engine 716 by comparing a cut path defined by the QA analysis engine 714 to a cut path defined by the processor computing device 130.

In some instances, the 3D model output data of QA samples can be used to adjust angled cut paths for workpieces using waterjet cutters. Angled cut paths may be needed to precisely remove features of a workpiece. For instance, fat, bones, or other undesirable material may run through workpieces at an angle. An angled cut is often required to cut away that undesirable material, such as fat or bones without cutting away valuable lean (meat) of the workpiece. In some instances, the 3D model output data of QA samples can include information regarding the angles of the internal features, and the 3D model output data can be used to adjust cutting paths of workpieces at the cutter station 122 based on estimated locations of an angled feature inside the QA sample. Adjustments may be made by the machine adjustment engine 716 by comparing a cut path defined by the QA analysis engine 714 to a cut path defined by the processor computing device 130.

Angled cut paths may also be needed to optimize downstream processing steps of workpieces. For instance, angled edges/faces can produce a workpiece having a higher surface area per weight, allowing for more breading pickup and/or improved appearance. The 3D model output of QA samples may be used to adjust target angled workpiece edges/faces based on the QA sample thickness/height, internal features, outline shape, classification, etc.

In some instances, the 3D model output data of QA samples can be used to predict voids, undercutting, or other irregularities of workpieces. In that regard, one or more workpiece anomaly machine learning models may be used to output a predicted workpiece shape, workpiece contour, or absence of substrate, including voids, undercutting, or other irregularities, based on a measured weight and volume (per QA image data) of one or more QA samples as input. In that regard, the one or more workpiece anomaly machine learning models may be trained using QA data regarding weight and volume, etc., of a QA sample correlated to observed or measured voids, undercutting, or other irregularities of the QA sample.

The workpiece anomaly machine learning model outputs may be used by the machine adjustment engine 716 to adjust any processing aspects of workpiece processing. For instance, if QA samples are measured to have a weight and volume lower than expected based on a density setting of processing system, one or more parameters or setting of the processing system may be adjusted, such as its density setting, a slice thickness, a portion size, etc., to account for the discrepancy.

In other examples, a conveyor 3D generation machine learning model may be configured to generate a 3D model of one or more components of the conveyance system 116, such as the powered conveyor belt 115. As noted above, images of the conveyance system 116 may be used to assess belt sag, belt wear, or other issues or information that can affect food product processing accuracy. The component 3D model may be compared to a specification for that component by the QA analysis engine 714. The component specification may include a CAD image of the component or a CAD image of the system containing the components (e.g., the entire conveyance system 116), optical images of the components/system, and/or measured values of components/system.

For instance, QA analysis of machine components, such as conveyor system components may include comparing measurements of belt components to a 3D model of the conveyance system 116. If a distance between belt pickets or rods in the 3D model is measured to be more than a distance in the specification, the QA analysis engine 714 may output such information to the machine adjustment engine 716, which may adjust processing to account for stretch in the conveyor belt. In other examples, such a comparison may be used to track gaps in the belt between pickets or rods, and compare the gap to the model to determine if/how the gap is changing over time. In other examples, measurements of belt links in the 3D model may be compared to previous measurements or known dimensions to account for sag/wear in the belt.

In other examples, an image segmentation machine learning model may be configured to identify features of a QA sample, identify separate portions of a sample, etc., by segmenting or “cutting out” an object, feature, etc., in an image as output based on QA image data as input. For instance, an image segmentation machine learning model may use still camera images to identify features of a QA sample. The image segmentation machine learning model may incorporate the Segment Anything Model (SAM) available from Meta AI, FastSAM from Ultralytics, or another suitable image segmentation model using image segmentation techniques.

In one example, a segmented image output identifies each of a plurality of related QA samples in QA image data. For instance, the segmented image output may include information (e.g., outlines in an image) for identifying each portion of a portioned workpiece (e.g., each chicken nugget from a chicken breast fillet). The identified related QA samples or portions of the workpiece may be correlated to an individual weight for each portion. As described above, a QA weight for each of a plurality of related QA samples may be obtained by adding or taking away each of the related QA samples on a weight platform, by weighing all the related QA samples together to obtain a total weight and then assigning a fraction of that total weight to each individual related QA sample (e.g., using individual volume: total volume, as determined from QA image data). The individual QA weight for each of the plurality of related QA samples may be used with the segmented image output to perform a QA analysis for each of the plurality of related QA samples.

On a related note, the QA analysis may include correlating or analyzing aspects of the workpiece used to create the related QA samples using the QA analysis results of each individual related QA sample. For instance, based on a QA analysis of chicken nuggets portioned from a chicken breast fillet (e.g., whether each of the nuggets are within a shape, weight, and/or size spec) certain aspects of the chicken breast fillet may also be determined. For instance, if the nuggets are determined to have a density higher than expected (such as determined by the weight and size), the density settings on the machine for portioning the chicken breasts may be adjusted, the incoming chicken breasts may be diverted for massaging before portioning, etc.

In some examples, a feature recognition image segmentation machine learning model may provide an outline of bones as output based on a still image from the image sensor assembly 132 as input. The output may be a binary image or a map showing the location of the bones, with every pixel indicating the presence or absence of bone. With accurate data regarding bone location in a QA sample, adjustments to the trimming, cutting, etc., of workpieces can be made to cut more closely to the bone, minimizing product waste or yield loss. Further, bone location may also be used by the QA analysis engine 714 to classify QA samples/workpieces. Such bone location data may be used alone or in combination with classification probability scores, as discussed above.

A fat/lean boundary image segmentation machine learning model may also be used to identify fat/lean boundaries in QA samples. For instance, the fat/lean boundary image segmentation machine learning model may provide an image having an outline of fat and/or lean areas in a QA sample as output based on an optical image(s) as input. The model output may be, for instance, a marked-up version of the input QA image with computer-generated annotations showing outlines of the fat and/or lean areas in the QA sample.

Although image segmentation models can be used without training, in some examples, the reliability and efficiency of the image segmentation machine learning model may be optimized by supplying training data to the model management computing device 113. For instance, annotated QA images or other QA sample images showing outlines of features, cut lines, etc., may be used to further train the image segmentation machine learning model.

In other examples, a region of interest (ROI) machine learning model may be configured to generate an ROI of a QA sample as output based on a QA image as input. The ROI may be a proposed portion or outline of an area/object of a QA sample. The ROI may be represented as a binary mask image (e.g., in the mask image, pixels that belong to the ROI are set to 1 and pixels outside the ROI are set to 0) or in another format usable by QA analysis engine 714. The model output may further include symbolic (textual) labels added to the ROI, such as to describe its content in a compact manner, as well as individual points of interest (POI) within the ROI.

The ROI in the QA image may be used to locate a feature in the QA sample, it may be used to designate an area in the QA sample for a measurement (e.g., a height measurement, a temperature measurement, etc.), or some other purpose. In some examples, the ROI output of a ROI machine learning model is used to define an area on a QA sample likely defining a peak thickness/height of the QA sample. For instance, chicken breast fillets are not uniform in thickness/height across the width/length of the chicken breast. Rather, a peak thickness/height of the chicken breast is typically at a rounded end of the chicken breast, and the slimmer part of the breast is near a pointy end of the breast. If part of a chicken breast is thicker than the other, it will take longer for the thicker part to get to a safe temperature during a cooking process. As the thicker part reaches the safe temperature, the slimmer part will dry out. Accordingly, adjustments to chicken breasts processing (e.g., portioning, trimming, sorting, etc.) made by made by the machine adjustment engine 716 in response to output of the QA analysis engine 714 indicating a discrepancy to account for the cooking temperature differences. To manage such processing, an accurate peak height measurement can be important.

An ROI indicating a peak height area of a chicken breast may include an area in a rounded end of the chicken breast (which is the thickest/tallest area of the breast) that is substantially level. By finding the flattest spot in a peak thickness region of the chicken breast, the ROI will likely exclude any ridges and meat protrusions. The ROI output may also include a POI indicating the precise peak height for the chicken breast. The QA analysis engine 714 may use the ROI/POI output of the ROI machine learning model to determine whether adjustments should be made to processing of the chicken breast.

In some examples, the ROI output of a ROI machine learning model may define an area on a chicken breast for measuring height and/or slope of a caudal ridge of a chicken breast or butterfly to check for woody chicken. As is known in the industry, woody chicken, or chicken that has an unpleasant texture (e.g., hard to the touch, tougher, more complex consistency, coarse fiber texture, etc.), can often be recognized by a prominent caudal ridge. If an ROI is identified in a QA image of the chicken pertaining to a relevant caudal ridge area, the relevant height/slop of the caudal ridge can be determined for grading/assessing the chicken. As an example, pieces with no detected woody chicken may be utilized for premium sandwich portions, while pieces with slight woodiness may be used for lesser valued thin sliced portions, and pieces with more extreme woodiness may be diverted to products often made from trim, such as pet foods or marinate solutions.

As noted above, the ROI output in the QA image may be used to locate a feature in the QA sample. In some examples, an ROI machine learning model may be used to locate an ROI in a piece of steak likely containing a sciatic nerve. The sciatic nerve, which can be found in filet mignon or other cuts of steak, is typically located within a layer of fat in the steak. Moreover, the sciatic nerve is often located within a center of a largest portion of a specific fatty region of the steak. In that regard, in some examples, the ROI output for a steak piece may be defined by a largest inscribing circle that can be superimposed onto the fatty region in the QA image.

A POI in the ROI may be at substantially the center of the ROI, locating the likely location of the sciatic nerve. The ROI/POI output may be sent to the QA analysis engine 714 for QA analysis of the steak. For instance, QA analysis may include verification of sciatic nerve removal, verification of sciatic nerve retention in a portioned piece of steak, etc. If, based on the QA analysis, processing of steak with a sciatic nerve is outside specifications for the steak as determined by the QA analysis engine 714, adjustments to steak processing may be made (e.g., through the machine adjustment engine 716).

An ROI machine learning model may be trained with QA image data and other QA sample image data identifying the region of interest, such as with annotations, labels, etc. The ROI machine learning model learns to identify the ROI based on the features recognized in the images compared to the training data and the location of the ROI/POI relative to those features. For instance, if a human operator is measuring chicken breasts at a QA station, the operator may indicate a peak height location in a QA image, such as with a touch screen. For the sciatic nerve, an image of a piece of steak may be annotated to include a largest inscribing circle in the specific layer of fat containing the nerve. Such annotated QA image data may be sent to the model management computing device 113 for training the ROI machine learning model.

Other machines learning models may be executed by the QA analysis engine 714 using QA data of a QA sample as input, such as to provide information regarding a carcass side of a QA sample, a skin side of a QA sample, a left and/or right side of a fillet, a tenderloin notch location, rib locations, etc.

Any suitable type of machine learning models may be used, including but not limited to convolutional neural networks. Any suitable technique may be used to train the machine learning models, including but not limited to one or more of gradient descent, data augmentation, hyperparameter tuning, and freezing/unfreezing of model architecture layers. In some examples, annotated, raw images and in some instances, weight data are used as the training input. In some examples, one or more features derived from the images, including but not limited to versions of the images in a transformed color space, set of edges detected in the image, one or more statistical calculations regarding the overall content of the images, or other features derived from the images may be used instead of or in addition to the annotated raw images to train the machine learning models.

The QA analysis engine 714 is configured to generate machine learning model output data by running one or more of the machine learning models discussed herein or other suitable models. The QA analysis engine 714 may perform any necessary post-processing of the outputs for use by the machine adjustment engine 716, the processor computing device 130, and/or another computing device.

For instance, the QA analysis engine 714 may include one or more formatting modules configured to perform, for instance, any of the pre-processing steps noted above or any other steps necessary for using the outputs in managing processing of a QA sample (e.g., matching the formatting of the output data to the original QA data, formatting the output data for compatibility with one or more modules of the machine adjustment engine 716, etc.). In one example, formatting modules may be configured to convert pixel locations associated with aspects of an output image to a coordinate system of the machine adjustment engine 716 and/or the processor computing device 130. Post-processing may also include digitizing or reducing the data for efficient data transfer between the QA computing device 111 and another computing device.

The QA analysis engine 714 may also include one or more modules configured to select one or more outputs of a plurality of outputs generated by the machine learning models. For instance, if the machine learning model outputs three possible classification labels for a QA sample (e.g., a type of sub-primal cut such as pork chops), each with varying degrees of probability, the QA analysis engine 714 may categorize the QA sample as a certain type based on information sent from the package optimization engine 718.

For instance, if the package optimization engine 718 sends information to the QA analysis engine 714 indicating that the supply of QA samples, e.g., pork chops, is likely to contain more of a certain type, then a QA sample that may be classified as one of multiple types (per product specifications) may be classified as the type of chop that is in lower supply. In the alternative or in addition thereto, if the package optimization engine 718 sends information to the QA analysis engine 714 indicating that the demand of certain workpieces, e.g., sirloin pork chops, is high, then a QA sample that may be classified as one of multiple types (per product specifications) may be classified as the type of chop that is in higher demand. In that manner, a production run profit can be maximized.

The QA analysis engine 714 may also include one or more modules configured to extract information from the machine learning model output data or other QA data for sending to the machine adjustment engine 716 and/or the processor computing device 130. For instance, the QA analysis engine 714 may receive a segmented image of a QA sample as an output of the machine learning model, and the QA analysis engine 714 may extract various parameters from the segmented image (e.g., position, size, aspect ratio, outline, etc.). For instance, as noted above, a segmented image output may identify each of a plurality of related QA samples in QA image data (e.g., nuggets of a chicken breast fillet), and the identified related QA samples may be correlated to an individual weight for performing a QA analysis of each of the related QA samples.

It should be appreciated that machine learning model outputs, and extracted data from machine learning model outputs, etc., in addition to QA images, QA weight data, other QA analysis data, etc., may be considered “QA data” as used herein.

The QA analysis engine 714 may send (optionally post-processed) QA data to the machine adjustment engine 716 and/or the workpiece processing engine 312 of the processor computing device 130 and/or save any QA data in the QA data store 720 for retrieval by the machine adjustment engine 716. The machine adjustment engine 716 and/or the workpiece processing engine 312 of the processor computing device 130 uses information in the post-processed output data to determine a next step(s), if any, for adjusting processing of the QA sample.

Exemplary aspects of the machine adjustment engine 716 will now be described. As noted above, the QA analysis engine 714 may send post-processed output data to the machine adjustment engine 716 and/or the workpiece processing engine 312 of the processor computing device 130. It should be appreciated that the machine adjustment engine 716 may be incorporated into the workpiece processing engine 312 of the processor computing device 130, and therefore when describing aspects of the machine adjustment engine 716, it should be appreciated that any function may instead be carried out by the workpiece processing engine 312 of the processor computing device 130.

The machine adjustment engine 716 may be configured to execute one or more machine adjustment modules to process QA data and determine what, if any, adjustments need to be made to processing steps or components. Generally, a machine adjustment module may be configured to provide information regarding an adjustment(s) to a workpiece processing system recommend or required based on a QA analysis of the QA analysis engine 714. The information may include instructions for display or retrieval by an operator of the processing system, instructions that, in response to execution of a controller of the processing system (e.g., the processor(s) 302 of the processor computing device 130), automatically or semi-automatically change a setting in the processing system 104 for processing workpieces, or other information relevant for adjusting or confirming aspects of a system or process used for processing workpieces.

In some examples, the machine adjustment engine 716 may run a density adjustment module configured to automatically or semi-automatically adjust a density setting on the processing system 104 if a measured density of a QA sample is out of spec. For instance, a density value on a machine may be adjusted to process workpieces based on actual density of the QA samples (e.g., changing a density setting from 1.0 to 1.2) rather than based on an estimated density of incoming workpieces. Adjusting a density setting on the processing system 104 may automatically adjust a processing setting of the system, such as how the cutter station 122 cuts, slices, or trims a workpiece to achieve a certain portion size, thickness etc.

In other instances, a processing setting of the machine may be adjusted to account for a density difference. For instance, cutting instructions for a waterjet cutter may be adjusted to adjust a size or shape of a portioned workpiece based on a density difference. In another example, a slicer may be adjusted to account for different density values of the workpiece (e.g., if a higher density is measured by the QA scanning system 108, a smaller slice may be made to achieve a slice within a weight spec).

In some examples, the machine adjustment engine 716 may run a thermal processing adjustment module configured to automatically or semi-automatically adjust a thermal processing setting on a thermal processing system if a measured size, height, etc. of a QA sample is out of spec. For instance, a temperature setting (and/or humidity setting and/or other thermal processing settings, such as belt speed) on a machine may be adjusted to ensure larger or smaller workpieces than expected are properly cooked, frozen, etc.

In some examples, the machine adjustment engine 716 may run a preventive maintenance module configured to determine appropriate machine part repair/replacement if a measured QA sample is out of spec, such as a conveyor belt part. For instance, if a machine part, per a QA analysis by the QA analysis engine 714, is out of spec by less than a certain percentage, the machine adjustment engine 716 may indicate that a repair is needed. If a machine part, per a QA analysis by the QA analysis engine 714, is out of spec by more than a certain percentage, the machine adjustment engine 716 may indicate that a replacement is needed, and automatic ordering and/or notification of a technician may occur.

In some examples, the machine adjustment engine 716 may run a simulation module configured to simulate processing of a workpiece after an adjustment, repair, etc., has been made to the processing system. For instance, the machine adjustment engine 716 may output simulation instructions to a processor or controller of a processing system (e.g., the processor(s) 302 of the processor computing device 130), which may execute the simulation and, for instance, display simulation results on a display of a computing device.

In some examples, the machine adjustment engine 716 may execute one or more machine learning models, e.g., stored in the QA model data store 724, configured to output one of information regarding an adjustment(s) to a processing system, instructions that, in response to execution of a controller of the processing system, automatically or semi-automatically change a setting in the processing system, or other information relevant for adjusting or confirming aspects of a system or process used for processing workpieces based on QA data as input.

For instance, one or more of the density adjustment module, the thermal processing adjustment module, and the preventive maintenance module may execute one or more machine learning models that provide information or instructions for adjusting a density setting or related setting, a thermal processing setting, or for repairing or replacing a machine component as output based on QA data as input. Such machine learning models may be trained using QA analysis data of the QA analysis engine 714 and information regarding machine adjustments made to a processing system based on the QA analysis data.

The machine adjustment engine 716 may instead or additionally run any suitable module and/or machine learning model to adjust a processing system to conform workpieces to the desired specification in response to analysis of the QA analysis engine 714.

The data normalization engine 717 of the QA computing device 111 will now be described. The data normalization engine 717 may be run to associate or normalize workpiece data across platforms, computing devices, etc. of the workpiece processing management system 102. Normalization of data may include correlating QA data to processing system data (e.g., X-ray data, optical scan data, temperature data, etc.) such that a processing system of the workpiece processing management system 102, such as processing system 104, may adjust machine settings and/or take corrective action based on processing system data alone. In other words, an X-ray image alone may be used to generate workpiece mass data based on previous correlations of mass data to workpieces having certain attributes in an X-ray image.

In that regard, one or more normalization data machine learning models may be used to normalize data by providing QA data as output based on processing system data as input. For instance, the machine learning model may process an X-ray scan from the processing system 104 as input, and based on an attribute of that X-ray scan (e.g., an analog signal associated with the scan, an outer perimeter of the workpiece determined from the scan, etc.), the machine learning model may output a mass of the workpiece. The machine learning models may be trained with correlations of QA data and processing system data.

Normalization of data will allow for QA data to be associated with processing system data for faster and optimal processing of workpieces. For instance, adjustments to a processing system may be made based solely on the processing system data rather than needing to obtain QA data. In that regard, a primary purpose of the QA station 106 may be used to train one or more machine learning models, including a normalization data machine learning model(s).

The package optimization engine 718 of the QA computing device 111 will now be described. The package optimization engine 718 may be run to analyze the models or other image data generated by the model generation engine 712 for determining how to most optimally use the QA samples/workpieces. For instance, if portioned pieces are within a certain degree of specification as determined by the QA analysis engine 714, such portioned pieces may be considered “higher value” pieces that may be instructed to be packaged together by the package optimization engine 718 to maximize a package value. In other instances, pieces that are within a certain degree of specification may be designated for packaging for a specified customer having stricter requirements (tighter specs), whereas other pieces may be designated for other packaging.

The package optimization engine 718 may run a global optimization to assign each piece or a certain quantity of pieces to a package configuration based on QA analysis results from the QA analysis engine 714. The package configuration assigned to each QA sample/workpiece, piece, or quantity of pieces may be based on information pertaining to a supply of raw, incoming workpieces, requirements of finished QA workpieces, or other information from other sources. The finished QA sample data may identify at least one of a monetary value and a demand for each packaging configuration. One or more machine learning models may be trained to identify a package configuration as output based on a QA analysis of QA samples (e.g., percentage within spec) as input. In that regard, performing the global optimization may include using one or more machine learning models to identify a package configuration for a QA sample(s)/workpiece(s).

Referring back to FIG. 1, the QA sample processing management system 102 further includes a monitoring system 112 which may be embodied in any computing device in networked communication with the processing system 104 and/or the QA station 106. The monitoring system 112 includes a processor(s) (not shown) and computer readable medium having logic stored thereon that, in response to execution by the processor(s), causes the monitoring system 112 to provide a QA data processing engine 142 and a QA analysis reporting engine 144.

The QA data processing engine 142 is generally configured to receive QA analysis data (such as from the QA analysis engine 714, the machine adjustment engine 716, and/or the package optimization engine 718) and analyze the QA analysis data. Analysis of the QA analysis data may include compiling information for a certain machine or production run (e.g., percentage of samples out of spec, how far out of spec, thermal processing data, etc.), comparing QA data between production runs, comparing QA data to machine scan data, etc. The QA analysis reporting engine 144 may be configured to report the QA analysis data by arranging the data and displaying the data to a viewer. In that regard, the monitoring system 112 is generally configured to monitor the QA process and provide insight into the QA process, such as for optimizing the QA process, identifying any gaps in the QA process, troubleshooting machine equipment or settings based on QA process results, etc.

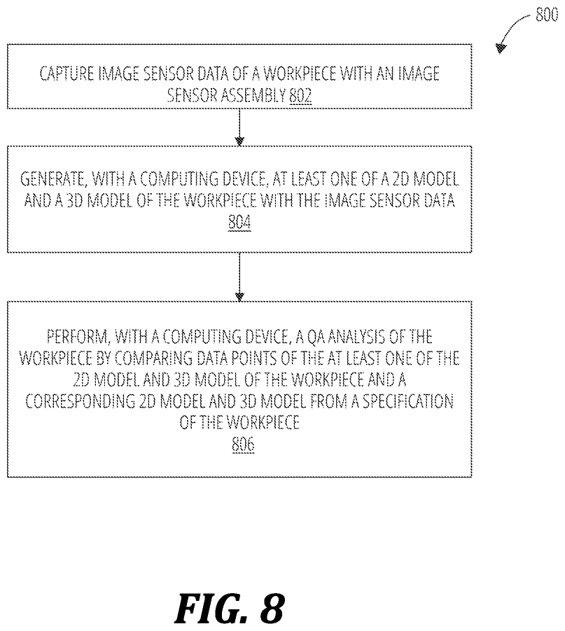

FIG. 8 is a flowchart that illustrates a non-limiting example of a method 800 of performing a quality assurance (QA) analysis for a QA sample, which may be carried out by one or more engines of the QA computing device 111 or any other computing device. The method 800 may be carried out for workpieces being processed by or to be processed by a processing system, such as processing system 104.

At block 802, the method 800 may include capturing image sensor data (e.g., one or more images) of a QA sample with an image sensor assembly. In some examples, the image sensor assembly is configured to generate at least one still color camera image and/or 3D point cloud data. For instance, the images of the QA sample may be captured using the image sensor assembly 132 described above, which may include at least one still camera and a structure light scanner system. Capturing image sensor data may include moving at least one image sensor of the image sensor assembly relative to the QA sample to capture an image of the QA sample at an oblique angle. The image sensor data may be captured by the image sensor assembly either when the QA sample is stationary or when the QA sample is being moved passed the image sensor assembly (such as with a conveyance system).

At block 804, the method 800 may include generating, with a computing device, at least one of a 2D model and a 3D model of the QA sample. For instance, the image processor 134 may send still camera image data and/or 3D point cloud data generated from the image sensor data to the image data processing engine 710 of the QA computing device 111. The image data processing engine 710 may generate a height map using the 3D point cloud data to create at least one of a 2D model and a 3D model of the QA sample. The image data processing engine 710 may run an image data optimization module to select 3D point cloud data from conflicting and/or competing sensor data originating from different image sensors and/or different sensor sources of the image sensor assembly. In some examples, one or more machine learning models may be used to identify 3D point cloud data from conflicting/competing sensor data as output based on at least one of an accuracy and efficiency of generating the model with the selected data as input.

At block 806, the method 800 may include performing, with a computing device, a QA analysis of the QA sample by comparing data points of the at least one of the 2D model and 3D model of the QA sample and a corresponding 2D model and 3D model from a specification of the QA sample. For instance, the QA analysis engine 714 may run a specification module that may include comparing data of the models or other image data generated by the model generation engine 712 to processing specification information for the QA sample. For instance, running the specification module may include identifying coordinates along the outer perimeter of the model and comparing those coordinates to specifications of the QA sample for assessing the shape, size, specific length/widths, etc., of the QA sample. If the data sets match within a fixed threshold level, then the specification module may indicate that the QA sample conforms to the product specification. If the data sets do not match within a fixed threshold level, then the specification module may indicate that the QA sample is out of spec, and in some instances, how far out of spec (e.g., the percentage of shape non-conformance, the percentage of length difference, etc.).

In some examples, the method 800 may further include capturing color sensor data of the QA sample with the image sensor assembly and performing, with a computing device (such as the QA analysis engine 714 of the QA computing device 111), a QA analysis of the QA sample by comparing the color sensor data of the QA sample captured with the image sensor assembly with color data values from at least one of a specification of the QA sample and a scan of the QA sample.

In some examples, the method 800 may further include obtaining a weight measurement of the QA sample with a weight measurement assembly, such as weight measurement assembly 110, and performing, with a computing device, a QA analysis of the QA sample by comparing at least one of the measured weight and a calculated density of the QA sample based on the measured weight with at least one of weight and density values determined from at least one of a specification of the QA sample and a scan of a workpiece of the same type.

In some examples, the method 800 may further include capturing color sensor data of the QA sample with the image sensor assembly and using one or more machine learning models to identify a consistency of the QA sample as output using one or more of color sensor data and the calculated density of the QA sample based on the measured weight of the QA sample as input.

In some examples, the method 800 may further include positioning the QA sample on a vertically displaceable surface, capturing one or more images with an image sensor assembly (such as image sensor assembly 132) that show a vertical displacement of the vertically displaceable surface caused by a weight of the QA sample, and processing the image data showing the vertical displacement of the vertically displaceable surface to obtain a weight measurement of the QA sample.

In some examples, the method 800 may further include executing one or more machine learning models to output a QA analysis using QA data as input. For instance, any of the exemplary machine learning models described herein as being configured to be carried out by the QA analysis engine 714 may be used.

In some examples, the method 800 may further include adjusting, with a computing device (such as with the machine adjustment engine 716 of the QA computing device 111), a setting of a machine (such as processing system 104) configured for processing QA samples/workpieces based on the QA analysis of the QA sample.

In some examples, the method 800 may further include performing, with a computing device (such as with the package optimization engine 718 of the QA computing device 111), a global optimization to assign QA samples/workpieces to a package configuration based on the QA analysis of the QA sample.

Although the example method 800 described above depicts particular operations, the sequence and/or combinations of operations may be altered without departing from the scope of the present disclosure. For example, some of the operations described may be performed in parallel or in a different sequence that does not materially affect the function of the method 800. In yet some examples, some of the operations described may be omitted. In other examples, different components of an example device or system may be used to implement the method 800. The method 800 may be carried out using any of the aspects disclosed herein.

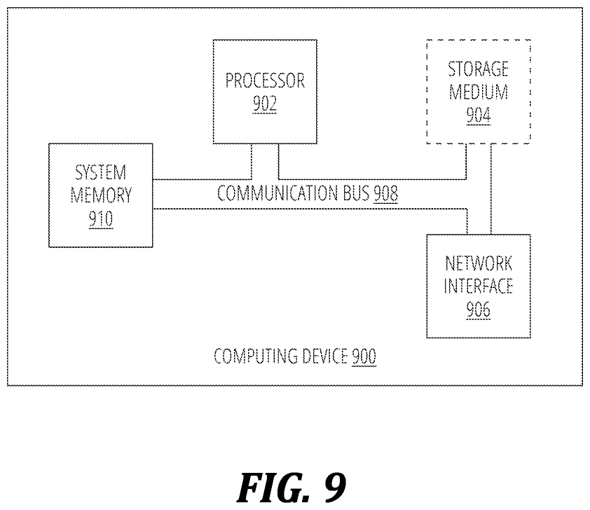

FIG. 9 is a block diagram that illustrates aspects of an exemplary computing device 900 appropriate for use as a computing device of the present disclosure. While multiple different types of computing devices were discussed above, the exemplary computing device 900 describes various elements that are common to many different types of computing devices. While FIG. 9 is described with reference to a computing device that is implemented as a device on a network, the description below is applicable to servers, personal computers, mobile phones, smart phones, tablet computers, embedded computing devices, and other devices that may be used to implement portions of examples of the present disclosure. Some examples of a computing device may be implemented in or may include an application-specific integrated circuit (ASIC), a field-programmable gate array (FPGA), or other customized device. Moreover, those of ordinary skill in the art and others will recognize that the computing device 900 may be any one of any number of currently available or yet to be developed devices.

In its most basic configuration, the computing device 900 includes at least one processor 902 and a system memory 910 connected by a communication bus 908. Depending on the exact configuration and type of device, the system memory 910 may be volatile or nonvolatile memory, such as read only memory (“ROM”), random access memory (“RAM”), EEPROM, flash memory, or similar memory technology. Those of ordinary skill in the art and others will recognize that system memory 910 typically stores data and/or program modules that are immediately accessible to and/or currently being operated on by the processor 902. In this regard, the processor 902 may serve as a computational center of the computing device 900 by supporting the execution of instructions.

As further illustrated in FIG. 9, the computing device 900 may include a network interface 906 comprising one or more components for communicating with other devices over a network. Examples of the present disclosure may access basic services that utilize the network interface 906 to perform communications using common network protocols. The network interface 906 may also include a wireless network interface configured to communicate via one or more wireless communication protocols, such as Wi-Fi, 2G, 3G, LTE, WiMAX, Bluetooth, Bluetooth low energy, and/or the like. As will be appreciated by one of ordinary skill in the art, the network interface 906 illustrated in FIG. 9 may represent one or more wireless interfaces or physical communication interfaces described and illustrated above with respect to particular components of the computing device 900.

In the example depicted in FIG. 9, the computing device 900 also includes a storage medium 904. However, services may be accessed using a computing device that does not include means for persisting data to a local storage medium. Therefore, the storage medium 904 depicted in FIG. 9 is represented with a dashed line to indicate that the storage medium 904 is optional. In any event, the storage medium 904 may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

Suitable implementations of computing devices that include a processor 902, system memory 910, communication bus 908, storage medium 904, and network interface 906 are known and commercially available. For ease of illustration and because it is not important for an understanding of the claimed subject matter, FIG. 9 does not show some of the typical components of many computing devices. In this regard, the computing device 900 may include input devices, such as a keyboard, keypad, mouse, microphone, touch input device, touch screen, tablet, and/or the like. Such input devices may be coupled to the computing device 900 by wired or wireless connections including RF, infrared, serial, parallel, Bluetooth, Bluetooth low energy, USB, or other suitable connections protocols using wireless or physical connections. Similarly, the computing device 900 may also include output devices such as a display, speakers, printer, etc. Since these devices are well known in the art, they are not illustrated or described further herein.

While the concepts of the present disclosure are susceptible to various modifications and alternative forms, specific examples thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one example,” “an example,” etc., indicate that the example described may include a particular feature, structure, or characteristic, but every example may or may not necessarily include that particular feature, structure, or characteristic. Moreover, such phrases are not necessarily referring to the same example. Further, when a particular feature, structure, or characteristic is described in connection with an example, it is submitted that it is within the knowledge of one skilled in the art to affect such feature, structure, or characteristic in connection with other examples whether or not explicitly described. Additionally, it should be appreciated that items included in a list in the form of “at least one A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Language such as “up”, “down”, “left”, “right”, “first”, “second”, etc., in the present disclosure is meant to provide orientation for the reader with reference to the drawings and is not intended to be the required orientation of the components or graphical images or to impart orientation limitations into the claims.

In the drawings, some structural or method features may be shown in specific arrangements and/or orderings. However, it should be appreciated that such specific arrangements and/or orderings may not be required. Rather, in some examples, such features may be arranged in a different manner and/or order than shown in the illustrative FIGS. Additionally, the inclusion of a structural or method feature in a particular FIG. is not meant to imply that such feature is required in all examples and, in some examples, it may not be included or may be combined with other features.

The present application may include modifiers such as the words “generally,” “approximately,” “about”, or “substantially.” These terms are meant to serve as modifiers to indicate that, for instance, the “dimension,” “shape,” “temperature,” “time,” or other physical parameter in question need not be exact, but may vary as long as the function that is required to be performed can be carried out.

As used herein, the terms “about”, “approximately,” etc., in reference to a number, is used herein to include numbers that fall within a range of 10%, 5%, or 1% in either direction (greater than or less than) the number unless otherwise stated or otherwise evident from the context (except where such number would exceed 100% of a possible value).

Where electronic or software components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The phrase “coupled to” refers to any component that is physically connected to another component either directly or indirectly, and/or any component that is in communication with another component (e.g., connected to the other component over a wired or wireless connection, and/or other suitable communication interface) either directly or indirectly.

Headings of sections provided in this patent application and the title of this patent application are for convenience only and are not to be taken as limiting the disclosure in any way.

While preferred examples of the present invention have been shown and described herein, it will be apparent to those skilled in the art that such examples are provided by way of example only. Numerous variations, changes, and substitutions will now occur to those skilled in the art without departing from the invention. Various alternatives to the examples of the invention described herein may be employed in practicing the invention. It is intended that the following claims define the scope of the invention and that methods and structures within the scope of these claims and their equivalents be covered.

### Listing of Innovations

Clause 1. A computer-implemented method of performing a quality assurance (QA) analysis for a QA sample that is at least one of processed by a processing system and to be processed by a processing system, the processing system having a controller configured for managing aspects for processing workpieces with the processing system in response to an analysis of the workpieces separate from the QA analysis, the method comprising: capturing QA image sensor data of a QA sample with an image sensor assembly of a QA system; generating, with a computing device, at least one of a 2D model and a 3D model of the QA sample using the QA image sensor data; and performing, with a computing device, a QA analysis of the QA sample by comparing QA data of the at least one of the 2D model and 3D model of the QA sample with a QA specification of the QA sample.

Clause 2. The computer-implemented method of clause 1, wherein the image sensor assembly includes at least one of stereo camera, a structure light scanner system, and a still camera.

Clause 3. The computer-implemented method of clause 1, further comprising moving at least one image sensor of the image sensor assembly relative to the QA sample to capture an image of the QA sample at an oblique angle.

Clause 4. The computer-implemented method of clause 1, wherein the QA specification of the QA sample is reference data, a 3D model, a drawing, or any combination thereof.

Clause 5. The computer-implemented method of clause 1, further comprising running, with a computing device, an image data optimization module to select QA image sensor data from at least one of conflicting and competing sensor data originating from at least one of different image sensors and different sensor data sources of the image sensor assembly.

Clause 6. The computer-implemented method of clause 5, further comprising selecting, with a computing device, the QA image sensor data depending on at least one of resolution of the image sensor data, processing power required to use the image sensor data, compatibility of the image sensor data with modules of the computing device, accuracy and/or efficiency of generating the model with the selected data as input.

Clause 7. The computer-implemented method of clause 1, further comprising using, with a computing device, one or more machine learning models to select QA image sensor data from at least one of conflicting and competing sensor data as output based on at least one of an accuracy and efficiency of generating the model with the selected image sensor data as input.

Clause 8. The computer-implemented method of clause 1, further comprising conveying the QA sample beneath the image sensor assembly with a conveyance system.

Clause 9. The computer-implemented method of clause 8, further comprising: conveying the QA sample over a weigh cell to obtain a QA weight measurement of the QA sample; and performing, with a computing device, a QA analysis of the QA sample by comparing at least one of the QA weight measurement and a calculated density of the QA sample based on the QA weight measurement with at least one of weight and density values determined from at least one of a QA specification of the QA sample and a scan of the QA sample by the processing system.

Clause 10. The computer-implemented method of clause 1, further comprising: obtaining a QA weight measurement of the QA sample with a weight measurement assembly; and performing, with a computing device, a QA analysis of the QA sample by comparing at least one of the QA weight measurement and a calculated density of the QA sample based on the QA weight measurement with at least one of weight and density values determined from at least one of a QA specification of the QA sample and a scan of the QA sample by the processing system.

Clause 11. The computer-implemented method of clause 10, further comprising adjusting, with a computing device, at least one of a density setting on the processing system, a cutting setting on the processing system, a sorting setting on the processing system, a packaging setting on the processing system, a temperature setting on the processing system, and a cooking time on the processing system when a QA sample has a calculated density different from the QA specification of the QA sample and the scan of the QA sample by the processing system.

Clause 12. The computer-implemented method of clause 10, further comprising obtaining a temperature of the QA sample or a workpiece during or after a thermal process when the QA sample or workpiece has a weight or volume exceeding a threshold weight or volume.

Clause 13. The computer-implemented method of clause 10, further comprising diverting at least one of a workpiece and the QA sample from a thermal process when the QA sample has a weight or volume exceeding a threshold weight or volume.

Clause 14. The computer-implemented method of clause 10, further comprising: capturing color sensor data of the QA sample with the image sensor assembly; and using one or more machine learning models to identify a consistency of the QA sample as output using one or more of color sensor data and the calculated density of the QA sample based on the QA weight measurement of the QA sample as input.

Clause 15. The computer-implemented method of clause 1, further comprising: capturing color sensor data of the QA sample with the image sensor assembly; and performing, with a computing device, a QA analysis of the QA sample by comparing the color sensor data of the QA sample captured with the image sensor assembly with color data values from at least one of a QA specification of the QA sample and a scan of the QA sample by the processing system.

Clause 16. The computer-implemented method of clause 1, further comprising: positioning the QA sample on a vertically displaceable surface; capturing one or more images with the image sensor assembly that show a vertical displacement of the vertically displaceable surface caused by a weight of the QA sample; and processing image data showing the vertical displacement of the vertically displaceable surface to obtain a QA weight measurement of the QA sample.

Clause 17. The computer-implemented method of clause 1, further comprising: scanning the QA sample with a scanning station of the processing system to produce scan data; generating at least one of a 2D model and a 3D model of the QA sample based on the scan data; and performing a QA analysis of the QA sample by comparing data points of a first data set containing the at least one of the 2D model and 3D model generated from the QA image sensor data of the QA sample and a second data set containing a corresponding 2D model and 3D model generated from the scan data of the QA sample.

Clause 18. The computer-implemented method of clause 17, performing a model match process to determine if the QA sample used to create the first data set is the QA sample used to create the second data set.

Clause 19. The computer-implemented method of clause 18, wherein the model match process includes performing translations of the first data set onto the second data set, wherein performing translations includes one or more of: directional translation of the QA sample; rotational translation of the QA sample; scaling a size of the QA sample; and shear distortion of the QA sample.

Clause 20. The computer-implemented method of clause 1, further comprising adjusting, with a computing device, a setting of the processing system based on the QA analysis of the QA sample.

Clause 21. The computer-implemented method of clause 1, further comprising performing, with a computing device, a global optimization to assign workpieces processed by the processing system to a package configuration based on the QA analysis of the QA sample.

Clause 22. The computer-implemented method of clause 1, further comprising outputting, with a computing device, at least one processing system setting adjustment as a list of possible adjustments based on the QA analysis of the QA sample.

Clause 23. The computer-implemented method of clause 1, further comprising executing, with a computing device, one or more machine learning models to output a QA analysis of the QA sample using at least one of the 2D model and 3D model of the QA sample as input.

Clause 24. The computer-implemented method of clause 23, wherein the one or more machine learning models, after receiving at least one of the 2D model and 3D model of the QA sample as input, are configured to perform at least one of: generating a 3D model of the QA sample; generating a classification probability score of at least one possible type of workpiece for the QA sample; generating a region of interest in an image of the QA sample; and generating an outline in an image of the QA sample of at least one object or feature of the workpiece.

Clause 25. The method of Clause 24, wherein generating a 3D model of the QA sample as output is in response to receiving images of first and second opposite surfaces of the QA sample as input.

Clause 26. The method of Clause 25, wherein the image of the first surface of the QA sample is an image of a top surface of the QA sample, and the image of the second surface of the QA sample is an image of a top surface of a prior cut QA sample.

Clause 27. The method of Clause 26, wherein generating a 3D model of the QA sample includes at least one of: extrapolating identified features from images of the top and bottom surfaces of the QA sample through a thickness of the QA sample; and extrapolating density data from a top surface of the QA sample down to a bottom surface of the QA sample to estimate a shape of the bottom surface including any voids.

Clause 28. The method of Clause 26, or 27, wherein the images of the top and bottom surfaces of the QA sample are height maps.

Clause 29. The method of Clause 26, 27, or 28, further comprising defining, for a processing system, cut paths of a workpiece based on features identified in the 3D model of the QA sample.

Clause 30. The method of Clause 27, or 28, wherein generating a classification probability score of at least one possible type of workpiece for the QA sample is based on at least one of the images of the top and bottom surfaces of the QA sample.

Clause 31. The method of Clause 30, wherein receiving and processing, by a computing device, the output of a classification probability score of at least one possible type of workpiece for the QA sample includes categorizing the QA sample based on at least one of first and second classification probability scores for the QA sample using the top and bottom surfaces of the QA sample, respectively, and using a demand for a first type of workpiece corresponding to the first classification probability score and a second type of workpiece corresponding to the second classification probability score.

Clause 32. The method of Clause 24, wherein generating a classification probability score of at least one possible type of workpiece for the QA sample includes at least one of: providing a label for the at least one possible type of workpiece for the QA sample if the classification probability score exceeds a minimum threshold; providing a list of first and second possible types of workpieces for the QA sample based on a first and second highest classification probability scores; and providing a list of every possible type of workpiece for the QA sample and corresponding classification probability scores for each type.

Clause 33. The method of Clause 24 or 32, wherein receiving and processing, by a computing device, the output of a classification probability score of at least one possible type of workpiece for the QA sample includes categorizing the QA sample based on at least one of the classification probability score and a demand for the at least one possible type of workpiece.

Clause 34. The method of Clause 33, further comprising performing, with the processing system, at least one of cutting, portioning, trimming, sorting, and packaging a workpiece based on the categorized type of the QA sample.

Clause 35. The method of Clause 24, wherein generating a region of interest in an image of the QA sample includes at least one of: superimposing a largest inscribing circle on an image of the QA sample in a fatty region of a steak likely to include a sciatic nerve; and superimposing an outline on an image of the QA sample defining a likely peak height portion of the QA sample.

Clause 36. The method of Clause 24, wherein the QA sample is a piece of chicken, and wherein generating a region of interest in an image of the piece of chicken includes at least one of: superimposing an outline on an image of the piece of chicken surrounding a substantially flat peak height portion of a chicken breast; and superimposing an outline on an image of the piece of chicken surrounding a portion of a caudal ridge of the piece of chicken for measuring height and/or slope of the caudal ridge relevant to assessment of woody chicken.

Clause 37. The method of Clause 24, wherein generating an outline in an image of the QA sample of at least one object or feature of the QA sample includes outlining at least one of each of a plurality of pieces of the QA sample, a bone(s), a fat/lean boundary, an edge of the QA sample, a perimeter of the QA sample, a bottom surface of the QA sample, and cut lines of the QA sample.

Clause 38. The computer-implemented method of clause 1, further comprising adjusting, with a computing device, at least one setting on the processing system when, based on the QA analysis, a QA sample has at least one of a physical parameter, characteristic, and attribute different than a corresponding physical parameter, characteristic, and attribute of a specification of the QA sample.

Clause 39. The computer-implemented method of clause 38, further comprising executing, with a computing device, one or more machine learning models to output machine adjustment instructions for adjusting a processing setting for processing workpieces using the QA analysis as input.

Clause 40. The computer-implemented method of clause 39, further comprising normalizing, with a computing device, QA data and workpiece processing system data.

Clause 41. The computer-implemented method of clause 40, further comprising executing, with a computing device, one or more machine learning models to output QA data as output based on processing system data as input.

Clause 42. The computer-implemented method of clause 1, further comprising defining, with a computing device, an imaging support surface plane that is substantially parallel to an imaging support surface on which the QA sample rests during imaging, wherein the imaging support surface plane may be used as a reference from which all height measurements for the QA sample may be determined.

Clause 43. The computer-implemented method of clause 42, further comprising: obtaining a QA weight measurement of the QA sample with a bench scale having a platform that defines the imaging support surface; and performing, with a computing device, a QA analysis of the QA sample by comparing at least one of the QA weight measurement and a calculated density of the QA sample based on the QA weight measurement with at least one of weight and density values determined from at least one of a specification of the QA sample and a scan of the QA sample by the processing system.

Clause 44. The computer-implemented method of clause 43, further comprising: obtaining, with the weight measurement assembly, a QA weight measurement for each of a plurality of related QA samples; capturing, with image sensor assembly, QA image sensor data of the plurality of related QA samples; identifying, with a computing device, each of the plurality of related QA samples in the QA image sensor data; and correlating, with a computing device, a QA weight measurement for each of the plurality of related QA samples to each of the identified plurality of related QA samples in the QA image sensor data; and performing, with a computing device, a QA analysis of each of the plurality of related QA samples by comparing at least one of the QA weight measurement and QA image sensor data with a specification of the QA sample.

Clause 45. The computer-implemented method of clause 44, further comprising performing, with a computing device, a QA analysis of each of the plurality of related QA samples by comparing at least one of the QA weight measurement and a calculated density of each of the plurality of related QA samples based on the QA weight measurement with at least one of weight and density values determined from at least one of a QA specification of each of the plurality of related QA samples and a scan of each of the plurality of related QA samples by the processing system.

Clause 46. The computer-implemented method of clause 44, wherein performing, with a computing device, a QA analysis of each of the plurality of related QA samples includes generating at least one of the 2D model and 3D model of each of the plurality of related QA samples.

Clause 47. The computer-implemented method of clause 44, wherein each of a plurality of related QA samples are portions of a workpiece.

Clause 48. A quality assurance (QA) system for performing a QA analysis of a QA sample that is at least one of processed by a processing system and to be processed by a processing system, the processing system having a controller configured for managing aspects for processing workpieces with the processing system in response to an analysis of the workpieces separate from the QA analysis, comprising: an image sensor assembly of a QA system configured to capture QA image sensor data of a QA sample; a processor; and a memory storing instructions that, when executed by the processor, cause a computing device of the QA system to: generate at least one of a 2D model and a 3D model of the QA sample; and perform a QA analysis of the QA sample by comparing QA data of the at least one of the 2D model and 3D model of the QA sample and a QA specification of the QA sample.

Clause 49. The QA system of clause 48, further comprising an image processor configured to generate at least one of 3D point cloud data and color data from the captured QA image sensor data of the QA sample.

Clause 50. The QA system of clause 48 or 49, wherein the image sensor assembly of the QA system includes at least one of stereo camera, a structure light scanner system, and a still camera.

Clause 51. The QA system of clause 48, further comprising a weight measurement assembly of the QA system configured to obtain a weight measurement of the QA sample by measuring a vertical displacement of an imaging support surface on which the QA sample is placed.

Clause 52. The QA system of clause 48, further comprising a weigh deck of the QA system configured to obtain a weight measurement of the QA sample and convey the QA sample past the image sensor assembly.

Clause 53. The QA system of clause 48, wherein the processing system has a scanning station configured to obtain one or more scans of a workpiece for generating at least one of a 2D model and a 3D model of the workpiece, wherein the memory of the computing device of the QA system further stores instructions that, when executed by the processor, cause a computing device of the QA system to perform a QA analysis of the QA sample by comparing QA data of the at least one of the 2D model and 3D model generated from the QA image sensor data of the QA sample and a corresponding 2D model and 3D model generated from the scan data of the workpiece.

Clause 54. The QA system of clause 48, wherein the QA specification of the QA sample is reference data, a 3D model, a drawing, or any combination thereof.

Clause 55. The QA system of clause 48, further comprising a conveyance system for conveying the QA sample beneath the image sensor assembly.

Clause 56. The QA system of clause 48, further comprising a weight measurement assembly of the QA system configured to capture a QA weight measurement of the QA sample.

Clause 57. The QA system of clause 56, wherein the weight measurement assembly is a bench scale having a platform, and wherein an imaging support surface of the image sensor assembly is defined by a top surface of the platform of the weight measurement assembly.

Clause 58. The QA system of clause 56, wherein the weight measurement assembly is defined by a vertically displaceable surface and the image sensor assembly, and wherein one or more images of the QA sample may be captured with the image sensor assembly that show a vertical displacement of the vertically displaceable surface caused by a weight of the QA sample such that a QA weight measurement of the QA sample may be obtained using the vertical displacement of the vertically displaceable surface.

Clause 59. The QA system of clause 48, further comprising a temperature measurement assembly of the QA system configured to capture a temperature measurement of the QA sample.

Clause 60. A computer-implemented method of performing a quality assurance (QA) analysis for a QA sample that is at least one of processed by a processing system and to be processed by a processing system, the processing system having a controller configured for managing aspects for processing workpieces with the processing system in response to an analysis of the workpieces separate from the QA analysis, the method comprising: capturing QA image sensor data of a QA sample with an image sensor assembly of a QA system; generating, with a computing device, at least one of a 2D model and a 3D model of the QA sample using the QA image sensor data; obtaining a QA weight measurement of the QA sample with a weight measurement assembly of the QA system; and performing, with a computing device, a QA analysis of the QA sample, including: comparing QA image data of the at least one of the 2D model and 3D model of the QA sample with a QA specification of the QA sample; and comparing at least one of the QA weight measurement and a calculated density of the QA sample based on the QA weight measurement with at least one of weight and density values determined from at least one of a QA specification of the QA sample and a scan of the QA sample by the processing system.

Clause 61. A computer-implemented method of performing a quality assurance (QA) analysis for a QA sample, the method comprising: capturing image sensor data of a QA sample with an image sensor assembly; generating, with a computing device, at least one of a 2D model and a 3D model of the QA sample using the image sensor data; and performing, with a computing device, a QA analysis of the QA sample by comparing QA data of the at least one of the 2D model and 3D model of the QA sample with a specification of the QA sample.

Clause 62. A quality assurance (QA) system for processing a QA sample, comprising: an image sensor assembly configured to capture image sensor data of a QA sample; a processor; and a memory storing instructions that, when executed by the processor, cause a computing device of the QA system to: generate at least one of a 2D model and a 3D model of the QA sample; and perform a QA analysis of the QA sample by comparing QA data of the at least one of the 2D model and 3D model of the QA sample and a specification of the QA sample.

The examples of the invention in which an exclusive property or privilege is claimed are defined as follows:

1. A computer-implemented method of performing a quality assurance (QA) analysis for a QA sample that is at least one of processed by a processing system and to be processed by a processing system, the processing system having a controller configured for managing aspects for processing workpieces with the processing system in response to an analysis of the workpieces separate from the QA analysis, the method comprising:
capturing QA image sensor data of a QA sample with an image sensor assembly of a QA system;
generating, with a computing device, at least one of a 2D model and a 3D model of the QA sample using the QA image sensor data; and
performing, with a computing device, a QA analysis of the QA sample by comparing QA data of the at least one of the 2D model and 3D model of the QA sample with a QA specification of the QA sample.

2. The computer-implemented method of claim 1, wherein the image sensor assembly includes at least one of stereo camera, a structure light scanner system, and a still camera.

3. The computer-implemented method of claim 1, further comprising moving at least one image sensor of the image sensor assembly relative to the QA sample to capture an image of the QA sample at an oblique angle.

4. The computer-implemented method of claim 1, further comprising running, with a computing device, an image data optimization module to select QA image sensor data from at least one of conflicting and competing sensor data originating from at least one of different image sensors and different sensor data sources of the image sensor assembly.

5. The computer-implemented method of claim 1, further comprising:
obtaining a QA weight measurement of the QA sample with a weight measurement assembly; and
performing, with a computing device, a QA analysis of the QA sample by comparing at least one of the QA weight measurement and a calculated density of the QA sample based on the QA weight measurement with at least one of weight and density values determined from at least one of a QA specification of the QA sample a scan of the QA sample by the processing system.

6. The computer-implemented method of claim 5, further comprising adjusting, with a computing device, at least one of a density setting on the processing system, a cutting setting on the processing system, a sorting setting on the processing system, a packaging setting on the processing system, a temperature setting on the processing system, and a cooking time on the processing system when a QA sample has a calculated density different from the QA specification of the QA sample and a scan of the QA sample by the processing system.

7. The computer-implemented method of claim 5, further comprising:
capturing color sensor data of the QA sample with the image sensor assembly; and
using one or more machine learning models to identify a consistency of the QA sample as output using one or more of color sensor data and the calculated density of the QA sample based on the QA weight measurement of the QA sample as input.

8. The computer-implemented method of claim 1, further comprising:
capturing color sensor data of the QA sample with the image sensor assembly; and
performing, with a computing device, a QA analysis of the QA sample by comparing the color sensor data of the QA sample captured with the image sensor assembly with color data values from at least one of a QA specification of the QA sample and a scan of the QA sample by the processing system.

9. The computer-implemented method of claim 1, further comprising:
positioning the QA sample on a vertically displaceable surface;
capturing one or more images with the image sensor assembly that show a vertical displacement of the vertically displaceable surface caused by a weight of the QA sample; and
processing image data showing the vertical displacement of the vertically displaceable surface to obtain a QA weight measurement of the QA sample.

10. The computer-implemented method of claim 1, further comprising executing, with a computing device, one or more machine learning models to output a QA analysis of the QA sample using at least one of the 2D model and 3D model of the QA sample as input.

11. The computer-implemented method of claim 10, wherein the one or more machine learning models, after receiving at least one of the 2D model and 3D model of the QA sample as input, are configured to perform at least one of:
generating a 3D model of the QA sample;
generating a classification probability score of at least one possible type of workpiece for the QA sample;
generating a region of interest in an image of the QA sample; and
generating an outline in an image of the QA sample of at least one object or feature of the workpiece.

12. The computer-implemented method of claim 1, further comprising adjusting, with a computing device, at least one setting on the processing system when, based on the QA analysis, a QA sample has at least one of a physical parameter, characteristic, and attribute different than a corresponding physical parameter, characteristic, and attribute of a specification of the QA sample.

13. The computer-implemented method of claim 12, further comprising executing, with a computing device, one or more machine learning models to output machine adjustment instructions for adjusting a processing setting for processing workpieces using the QA analysis as input.

14. The computer-implemented method of claim 1, further comprising:
defining, with a computing device, an imaging support surface plane that is substantially parallel to an imaging support surface on which the QA sample rests during imaging, wherein the imaging support surface plane may be used as a reference from which all height measurements for the QA sample may be determined;
obtaining a QA weight measurement of the QA sample with a weight measurement assembly defined by a bench scale having a platform that defines the imaging support surface; and
performing, with a computing device, a QA analysis of the QA sample by comparing at least one of the QA weight measurement and a calculated density of the QA sample based on the QA weight measurement with at least one of weight and density values determined from at least one of a specification of the QA sample and a scan of the QA sample by the processing system.

15. The computer-implemented method of claim 14, further comprising:
obtaining, with the weight measurement assembly, a QA weight measurement for each of a plurality of related QA samples;
capturing, with image sensor assembly, QA image sensor data of the plurality of related QA samples;
identifying, with a computing device, each of the plurality of related QA samples in the QA image sensor data;
correlating, with a computing device, a QA weight measurement for each of the plurality of related QA samples to each of the identified plurality of related QA samples in the QA image sensor data; and
performing, with a computing device, a QA analysis of each of the plurality of related QA samples by comparing at least one of the QA weight measurement and QA image sensor data with a specification of the QA sample.

16. The computer-implemented method of claim 15, further comprising performing, with a computing device, a QA analysis of each of the plurality of related QA samples by comparing at least one of the QA weight measurement and a calculated density of each of the plurality of related QA samples based on the QA weight measurement with at least one of weight and density values determined from at least one of a QA specification of each of the plurality of related QA samples and a scan of each of the plurality of related QA samples by the processing system.

17. The computer-implemented method of claim 15, wherein performing, with a computing device, a QA analysis of each of the plurality of related QA samples includes generating at least one of the 2D model and 3D model of each of the plurality of related QA samples.

18. A quality assurance (QA) system for performing a QA analysis of a QA sample that is at least one of processed by a processing system and to be processed by a processing system, the processing system having a controller configured for managing aspects for processing workpieces with the processing system in response to an analysis of the workpieces separate from the QA analysis, comprising:
an image sensor assembly of a QA system configured to capture QA image sensor data of a QA sample;
a processor; and
a memory storing instructions that, when executed by the processor, cause a computing device of the QA system to:
generate at least one of a 2D model and a 3D model of the QA sample; and
perform a QA analysis of the QA sample by comparing QA data of the at least one of the 2D model and 3D model of the QA sample and a QA specification of the QA sample.

19. The QA system of claim 18, further comprising a weight measurement assembly of the QA system configured to capture a QA weight measurement of the QA sample.

20. A computer-implemented method of performing a quality assurance (QA) analysis for a QA sample that is at least one of processed by a processing system and to be processed by a processing system, the processing system having a controller configured for managing aspects for processing workpieces with the processing system in response to an analysis of the workpieces separate from the QA analysis, the method comprising:
capturing QA image sensor data of a QA sample with an image sensor assembly of a QA system;
generating, with a computing device, at least one of a 2D model and a 3D model of the QA sample using the QA image sensor data;
obtaining a QA weight measurement of the QA sample with a weight measurement assembly of the QA system; and
performing, with a computing device, a QA analysis of the QA sample, including:
comparing QA image data of the at least one of the 2D model and 3D model of the QA sample with a QA specification of the QA sample; and
comparing at least one of the QA weight measurement and a calculated density of the QA sample based on the QA weight measurement with at least one of weight and density values determined from at least one of a QA specification of the QA sample and a scan of the QA sample by the processing system.
